(19) United States
(12) Patent Application Publication
(10) Pub. No.: US 2006/0198533 A1
(43) Pub. Date: Sep. 7, 2006

(54) Method and system for continuous monitoring and diagnosis of body sounds

(76) Inventors: Le Yi Wang, Novi, MI (US); Hong Wang, Novi, MI (US)

Correspondence Address: Le Yi Wang, 45466 Emerald Forest Drive, Novi, MI, 48374, US

(21) Appl. No.: 11/367,807

(22) Filed: Mar. 3, 2006

Related U.S. Application Data
(60) Provisional application No. 60/658,200, filed on Mar. 4, 2005.

(51) Int. Cl. A61B 7/04; H04B 15/00

(52) U.S. Cl. 381/67; 381/94.1

(57) ABSTRACT

A method and system is invented for automated continuous monitoring and real-time analysis of body sounds. The system embodies a multi-sensor data acquisition system to measure body sounds continuously. The sound signal processing functions utilize a unique signal separation and noise removal methodology by which authentic body sounds can be extracted from cross-talk signals and in noisy environments, even when signals and noises may have similar frequency components or statistically dependent. This method and system combines traditional noise canceling methods with the unique advantages of rhythmic features in body sounds. By employing a multi-sensor system, the method and system perform cyclic system reconfiguration, time-shared blind identification and adaptive noise cancellation with recursion from cycle to cycle. Since no frequency separation or signal/noise independence is required, this invention can provide a robust and reliable capability of noise reduction, complementing the traditional methods. The invention further includes a novel method by which pattern recognition of groups of key parameters can be used to diagnosis physical conditions associated with body sounds, with confidence intervals on the diagnostic criterion to indicate accuracy of diagnosis.

We hereby claim priority of provisional patent Ser. No. 60/658,200 filed on Mar. 4, 2005

### FIELD OF THE INVENTION

The present invention relates to a method and system for facilitating continuous monitoring, real-time analysis, and computerized diagnosis of body sounds. The system is able to record the body sounds, replay them, display them graphically, and store them for future utility. Based on acoustic data, or in combination with other monitored physiological signals, the system will perform evaluation of medical symptoms and diagnosis of medical disorders. The invention is especially targeted to, but not limited to, heart and lung sound monitoring, airway monitoring, analysis of lung, heart, and other body sounds for their related pulmonary, cardiac, and other related functions and disorders. In this invention, the term “body sound” is used to represent collectively sounds collected from parts of the animate body, such as sounds of respiration, heart, upper airways, snoring, voice, bowel, pulse and blood flows, including those of unborn children of pregnant women.

### BACKGROUND OF THE INVENTION

### Continuous Monitoring of Body Sounds

Body sounds contain a rich reservoir of vital physiological and pathological information. This information is useful for monitoring a person's physical conditions, and of critical importance for diagnosis and patient management, and vital sign monitoring for soldiers on the battlefield. Currently, heart and lung sound auscultation is routinely used in all clinical settings by healthcare providers using stethoscopes. However, the utility of the conventional stethoscope is limited to intermittent and manual auscultation by a healthcare worker, one auscultation site at a time.

### Noise Cancellation

Continuous monitoring of body sounds is of essential importance in medical diagnosis for patients with lung, cardiac, and sleep disorders and in detection of critical conditions in operating rooms. To obtain quantitative and reliable diagnosis and detection, it is critically important that body sound acquisition obtains sounds of high clarity. But clinical and out-patient acoustic environments impose great challenges for body sound acquisition. Unlike acoustic labs in which noise levels can be artificially controlled and reduced, and body sounds can be processed off-line, operating rooms and out-patient environments are very noisy due to surgical devices, ventilation machines, conversations, machine alarms, and other real-life noise artifacts. The unpredictable and broadband natures of such noises render these locations very difficult acoustic environments. Since body sounds cannot be directly controlled, and noises come from many sources and cannot be measured directly at their sources, separating body sounds from noises is a difficult blind source extraction problem.

Techniques for canceling off-band or statistically independent noises can be quite effective. The former can be attenuated by designing appropriate band-pass filters (frequency separation), and the latter by adaptive noise cancellation (ANC) (statistical separation). However, in actual practice, when noises are overlapping with the signal frequency bands of the body sounds, direct filtering can no longer eliminate the noises. Also, when noises correlate with lung sounds, they introduce a fundamental identification bias on the channel model that cannot be easily removed. Consequently, this model bias causes a decrease in quality of noise cancellation, rendering traditional adaptive noise canceling techniques ineffective. New methods of noise cancellation are therefore needed which are capable of performing beyond conventional frequency filtering and statistical techniques in order to handle more complex problems which arise in real situations requiring body sound measurement.

### Separation of Body Sounds

Body sounds, such as heart and lung sounds, interact with each other during auscultation, corrupting sound qualities and causing difficulty for diagnosis. For example, the main frequency components of heart sounds are in the range of 20-100 Hz, which often produce an intrusive interference that masks the clinical interpretation of lung sounds over the low frequency band. Therefore it is highly desirable, especially in computerized cardiopulmonary sound analysis, to separate the overlapping heart and lung sounds before using them for diagnosis.

### Pattern Recognition and Diagnosis

Body sounds are members of the group of physiological vital signs, which includes among others, heart rate, blood pressures, and oxygen saturation. Such signals contain a rich reservoir of vital physiological and pathological information that is of critical importance for clinical diagnosis and treatment management. More advanced technologies, such as X-ray, CT-scan, MRI, transesophageal echocardiogram (TEE), angiography, and ultrasonography, also have extensive diagnostic capabilities for physiological functions. However, the latter group cannot be routinely used in operating rooms or out-patient services due to their cumbersome testing equipment, complicated procedures, and difficulties in performing them in a continuous fashion. In contrast, continuous monitoring of body sounds could provide a non-invasive and inexpensive means of diagnosing accurately and promptly in many clinical conditions, such as misplaced intubation tubes, asthma, pulmonary edema, and detecting critical or even life-threatening situations such as airway obstruction, or clasp of lungs.

Assisted by standard engineering tools for signal processing, the fundamental characteristics of pulmonary sound waveforms can be extracted, classified, and employed to detect specific adventitious sound patterns and analyze their pathological implications. These findings have led to many publications on computer-aided detection of asthma, fibrosis and obstructive lung diseases, asbestosis, and heart failure. Numerous research groups have investigated potential computer-assisted lung sound analysis and classification.

However, it is well understood in pulmonary medicine that there are no universal sound patterns or parameter thresholds that definitively indicate a disease or medical condition. Individualized pattern recognition that combines information from sounds and other measurements needs to be established that is capable of capturing pattern shifting in each individual patient. To advance the frontier in computer-aided body sound analysis to real-life applications, new methods are needed to develop individualized pattern recognition techniques.

### DESCRIPTION OF THE PRIOR ART

### Prior Art in Body Sound Auscultation

Body sound auscultation has been performed by healthcare providers by using a stethoscope for more than 100 years. While stethoscopes have improved in their sound quality over the years, their fundamental pattern of usage remains unchanged: they are used to manually listen to body sounds intermittently and one site at a time.

### Prior Art in Noise Cancellation Using Filtering

Noise artifacts are well known to present a fundamental challenge towards developing automated lung and heart sound analysis. Conventional stethoscopes use a bell structure to block some noises and an acoustic chamber to amplify sounds. Improvement on stethoscopes has introduced more advanced electronic stethoscopes. In these electronic stethoscopes, basic frequency filtering is used remove noise whose frequency components are off the general frequency band of heart or lung sounds.

Traditionally, studies of heart and lung sounds have concentrated on filtering techniques. For example, it has been shown that in some selected cases inspiration, expiration, and first and second heart sounds are in different frequency bands [5,7,10,11]. Such distinctive features have then been used to design appropriate filters to extract useful signals. To further enhance the performance of the filtering process, FFT, power spectrum density, bi-spectrum analysis, wavelet analysis, high-order statistics, and stochastic averaging have been investigated extensively for their effectiveness in noise filtering and sound separation [6,7,8,9,11,14,15,16].

While off-band noises (those with frequencies outside the signal frequency band) can be easily filtered by band-pass filters, in-band noises (those whose frequencies overlap with the signal frequency band) are much harder to eliminate. As a result, in practice when noises are overlapping with the frequency band of the lung or heart sound, direct filtering methods can no longer eliminate the noises.

### Prior Art in Noise Cancellation using ANC

The problems of blind separation or blind extraction of source signals from noisy environments have received wide attention in various fields such as biomedical signal analysis and processing, geographical data processing, speech and image recognition, and wireless communications [12,18]. Although their underlying principles and approaches are different, most of these techniques are based on the classic principles of adaptive noise cancellation (ANC). The ANC approach usually reduces the noise by using reference signals, which give information about the noise interference acting on the observed data [13]. Since ANC does not require frequency-band separation as most classical frequency filtering methods rely upon, it provides an efficient noise cancellation method when signal/noise have overlapping frequency bands but are independent statistically. In other words, it is efficient in canceling the in-band noise, which would be impossible to obtain by using direct noise filtering.

However, the ANC method is based on the constraint that the noise signals be statistically independent from the source signals. In practice, this condition is often not satisfied. Therefore, ANC encounters significant challenges when the signal and noise are not independent or the underlying processes are not stationary.

### Prior Art in Separation of Heart and Lung Sounds

Much effort has been made in reducing heart/lung sound interference [1,2,3,4]. These methods all depend on distinctive features of heart and lung sounds to separate them for diagnosis of specific diseases. Most commonly, frequency separation features, statistical independence, or distinctive parameters are used. As a result, these techniques are disease specific.

### Prior Art in Pattern Analysis and Diagnosis

Dating from the invention of the stethoscope by Rene Laennec in 1816, a large number of systems have been invented which use noninvasive sensors to diagnose patient health. Similar to the stethoscope, other prior art in diagnostic medical systems is specifically designed for determination of lung functions. Some are tools which extract multiple parameters from a single sensor type. Lynn, et al, in U.S. Pat. No. 6,748,252, uses a pulse oximeter for generating a time series of oxygen saturation values and a set of frequency components to detect the occurrence of clustered variations indicative of clinically significant airway instability. But, in order to extract accurate frequency components, it is important to have signals free from noise.

A number of sensor types have been tried as substitutes for capturing lung sounds for diagnosis. For example, Casscells, III, et al, in U.S. Pat. No. 6,821,249, are able to use both the analysis of the speed and pattern of temperature changes as an indicator to determine worsening health conditions in patients with congestive heart failure. But problems of interpretation of results arise since these measured parameters are much less direct in relating to cardiac and pulmonary conditions than heart and lung sounds.

To improve direct measurements of body sounds, attempts have been made to improve upon the stethoscope, for example by adding ancillary parts to it. Thierman, in U.S. Pat. No. 6,790,184, adds a mechanical taper onto the end of the stethoscope which can aid the physician during the percussion portion of a physical exam. But this invention, while it will elicit more repeatable sounds, does not actually aid in capturing better un-stimulated chest sounds.

The importance of achieving a better way of recording respiratory sounds is evidenced by Derksen, et al., in U.S. Pat. No. 6,659,960, which discloses a portable unit for recording the upper airway respiratory sounds of an exercising horse to determine whether the horse suffers from an upper airway obstruction condition. But, all of these inventions still suffer from contamination by external noises and overlap in both frequency and time domains of other body sounds.

A number of prior inventions diminish the problem of reliance on noise free data from any one sensor by employment of a multiplicity of various sensor types. Prior art in diagnostic medical systems does include systems which combine outputs from a number of disparate sensors. Westbrook, et al., in U.S. Pat. No. 6,811,538, combines pulse, oximetry, snoring sounds, and head position of a patient to detect a respiratory event, such as sleep apnea. Other medical diagnostic systems also use multiple sensors.

However, these previous systems for the most part cannot build a personal model for diagnostics so that their diagnostic criteria are based upon averaged population models, rather than individual patient characteristics. Some inventions try to ameliorate this problem by personalizing the diagnostic process on the basis of eliciting the patient's personal satisfaction. While these opinion-in-the-loop systems could be personalized to preferences of a particular patient, they would still suffer from the lack of objective values from the patient in being able to assess what are really physiological and quantitative values. Iliff, in U.S. Pat. No. 6,770,029, invented a method for allowing a patient to perform disease management by using periodic interactive dialogs to obtain, among other information, health state measurements from the patient. Much use is made of the patient's preferences for treatment so that in addition to objective health measurements, subjective opinions enter the metric which is used to adjust patient therapy.

U.S. Pat. No. 6,701,271 to Willner, et al describes a system and method for using physical characteristic information obtained from two or more subjects to determine an evaluation of the data as well as a course of action to take with the subjects. In this case, an attempt to overcome the inadequacies of the ability of existing techniques to recommend a course of action based on an individual patient's characteristics is surmounted by averaging with other patients. This invention illustrates the need for better methods which can combine multi-sensor data along with other key parameters into an index which can give confidence values for the generated quantitative diagnosis on an actual individual basis.

A number of inventions disclose means to generate lung sound diagnostic prognostications for individual patients. For example, Murphy, in U.S. Pat. No. 6,790,183, discloses a lung sound diagnostic system which organizes and formats the lung sound data into a display for both inspiration and expiration combined in time scale. In a second display element, the data for inspiration and expiration are shown individually in a second time scale that is time-expanded relative to the first time scale. The system also provides for application programs to detect and classify abnormal sounds. The invention of Murphy includes an analysis program for comparing selected criteria corresponding to the detected abnormal sounds with predefined thresholds in order to provide a likely diagnosis. But U.S. Pat. No. 6,790,183 relies on display techniques and noise cancellation technologies, as described above, which are known to fail under realistic conditions. Moreover, even if the signals happened to be sufficiently free of external noise, the simple determination of diagnosis based on thresholds of predefined levels would not achieve an individualized treatment but an averaged value based on multiple patients of a population class.

All of the above mentioned prior systems are therefore deficient in their ability to serve as a platform for diagnosis of an individual patient's body sounds. Prior art does not provide means for noise cancellation or body sound separation which can remove noises with overlap in frequency, or separate out signals with similar statistics, or separate different body sounds under cross interference. Prior systems use means for making their diagnosis based on thresholds that are derived from patient populations, but do not provide a means to generate individualized diagnoses. No prior art is able to put confidence values on the diagnoses they determine. Likewise no prior art has provision for real-time tracking of the diagnostic variables, and moreover, none provide means for continuous updating of their underlying diagnostic algorithms.

### OBJECTS OF THE INVENTION

In view of the above state of the art, the present invention seeks to realize the following objects and advantages.

It is a primary object of the present invention to provide a method and system for monitoring multiple sites of body sounds automatically and continuously, playing and displaying sounds in audio and in graphical forms, and diagnosing body conditions and diseases.

It is another object of the present invention to provide a method and system with means for the cancellation of noise that has overlapping frequency components with the target signal.

It is another object of the present invention to provide a method and system with means for the cancellation of noise that is statistically correlated with the target signal.

It is another object of the present invention to provide a method and system with means for separation of body sounds which have similar stochastic and frequency features to the target signal.

It is another object of the present invention to provide a method and system with means for cyclic reconfiguration of signal transmission channels on the basis of the rhythmic nature of body sounds, such as heart beats and the inhale/exhale cycle in lung sounds, to identify the sound transmission channels iteratively, in real-time to separate heart and lung sounds and to remove undesirable noise artifacts.

It is another object of the present invention to provide a method and system with means for time-shared and individualized noise cancellation whereby the noise cancellation algorithm uses the phased nature of the body sounds to perform channel identification and noise cancellation.

It is another object of the present invention to provide a method and system with means for real-time and individualized adaptive pattern extraction that rates its quality in terms of confidence criteria.

It is another object of the present invention to provide a method and system with means for optimized dynamic diagnosis.

It is another object of the present invention to provide a method and system with a recursive noise cancellation process so that the algorithm can adjust itself to changes in the patient, environment, or both in real-time and with the minimum computational requirements.

It is another object of the present invention to provide a method and system with a recursive pattern extraction process so that the algorithm can adjust itself to changes in the patient, environment, or both in real-time and with the minimum computational requirements.

It is another object of the present invention to provide a method and system with a recursive optimized dynamic diagnosis process so that the algorithm can adjust itself to changes in the patient, environment, or both in real-time and with the minimum computational requirements.

It is another object of the present invention to provide user interface software which captures the pattern recognition information and displays the motion of the pattern in real-time on the computer screen. Thus, this invention allows the operator to follow the path of extracted key parameters with an electronic image display on the computer monitor.

It is also an object of the present invention to provide a multiple-sensor-based system that can be used to acquire lung and heart and other body sounds that can approximate distributed noises as a lumped noise source and perform signal separation and noise cancellation.

It is another object of the present invention to provide affirmation for successful restriction of key parameters to a normal region, warning alarms for deviation of key parameters from their safe regions, and remedial recommendations based on the automated parameter trajectories.

It is a further object of the present invention to provide a portable noise cancellation and pattern tracking device that can be interfaced to conventional personal computers and is fully automated and provides means for robust diagnostic functions beyond those of a simple sensor recording device, digital stethoscope, or Holter monitor.

These and other objects and advantages of the present invention will become more apparent from the description and claims which follow, or may be learned by the practice of the invention.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### SUMMARY OF THE INVENTION

This application claims the benefit of Provisional Patent Application No. 60/658,200 filed on Mar. 4, 2005.

This invention is based on the technology described with details in the references [19,20,21,22,23,24,25,26,27,28].

This invention introduces a monitoring system that is equipped with multiple acoustic and other sensors attached to multiple sites of a person's body to acquire body sounds and signals simultaneously and continuously. The system is capable of performing signal separation, noise cancellation, and computer-assisted signal pattern analysis. Based on acoustic sensor data, the system provides a non-invasive means of diagnosing accurately and promptly for many physical and clinical conditions, such as lung functions, heart rhythms, misplaced intubation tubes, asthma, pulmonary edema, airway obstruction, or clasp of lungs; and of keeping sound records for longitudinal analysis of disease progress and effectiveness of drug and procedures.

This invention presents a new technique for extracting authentic heart, lung, and other body sounds when those acquired sounds contain interference and noise corruption. Unlike many existing blind signal separation algorithms, which employ signal independence as the key mechanism of separation, the approach of the present invention utilizes the unique cyclic nature of cardiopulmonary sounds to conduct channel identification, signal separation, and noise cancellation iteratively. The algorithm reconfigures the signal transmission channels during different phases of breathing and heart beating cycles, thereby translating a difficult blind adaptive noise cancellation problem into a sequence of regular identification and noise cancellation problems. The reduced complexity problems are much easier to process and allow much faster convergence rates and less computational burdens. The techniques of the present invention are capable of identifying channel dynamics in real time, removing noise effectively, and separating heart and lung sounds. Consequently, computer-assisted analysis and diagnosis of heart, lung, and other body sounds can become more accurate and reliable than supported by prior art. One significant advantage of this invention is that it does not require signals and noises to possess the above separating features such as frequency separation, stochastic independence, or distinctive parameters. As a result, this invention is more generic and can be applied to a broader spectrum of application areas as long as the source signals non-synchronously go through existence and almost non-existence stages in cycles. It should be emphasized that this new method of the present invention can be used in combination with the prior existing techniques if the signals and noises do possess some desirable separation features, and hence this invention enhances the existing methodologies.

When used for processing only one type of body sound, such as lung sounds or heart sounds, this invention provides an improved methodology for extracting the authentic body sounds from noise-corrupted measurements. Unlike traditional noise cancellation methods that rely on either frequency band separation or signal/noise independence to achieve noise reduction, the methodology of the present invention combines the traditional noise canceling methods with the unique feature of time-split stages in body sounds, such as breathing cycles. By employing a multi-sensor system, the invention method first uses a band-pass filter to eliminate the off-band noise, and then performs time-shared blind identification and noise cancellation with recursion from breathing cycle to cycle.

In summary, the present invention consists of (1) a new multi-sensor monitoring system for body sounds; (2) new signal processing technologies for automatically and continuously eliminating noise artifacts due to noisy environments in, among others, hospitals, clinics, houses, or fields; (3) new signal processing technologies for automatically and continuously attenuating or eliminating signal interference among body sounds including, among others, lung, heart and upper airway sounds; (4) generic feature derivation algorithms that extract key parameters from waveforms of vital signs; (5) individualized and quantitative pattern recognition techniques to derive optimized pattern recognition regions that minimize decision errors; (6) individualized diagnosis algorithms for specific disorders with quantitative confidence levels of diagnosis accuracy; (7) computer display functions that demonstrate the past medical conditions, current diagnosis, and near-future predictions of a patient's symptoms and disorders.

### Multi-Sensor Body Sound Monitoring System

The system of the invention includes a sound acquisition module which consists of several sound sensors for measuring body sounds continuously and data acquisition unit, that is connected to a computing device. For convenience of operation and transport, all the hardware systems may be embedded in one overall system unit.

The acoustic sensors can be of any types that are sufficiently sensitive to acquire body sounds. These may include, but are not limited to, electronic stethoscopes, microphones, accelerometers, or special-purpose body sound sensors. The sensors will be attached to the designated auscultation sites and noise reference locations. In order to obtain noise measurements that represent the lumped impact of distributed and multi-source noises from the heart, lung, and other sound sensors, the noise reference sensors will be placed in the vicinity of the sound sensors. Some of the types of acoustic sensors require amplifiers to enhance sensitivity and signal/noise ratios. In these cases, amplifiers will be either connected to the sensors or embedded with the sensors in compact packaging. The outputs of the sensors will be connected to the data acquisition unit through signal wire interfacing, analog or digital, such as serial ports, USB ports, or wireless connections.

The main software is embodied in a Body Sound Analyzer Processing System that contains all the modules for processing vital sign signals. The signals are first conditioned and synchronized by the “Data Acquisition” module. To obtain authentic lung sounds, signals are filtered to remove off-band and independent noises by the “Filtering” module and “ANC” module. A new advanced noise cancellation technique, embodied in the module “Time Shared Noise Cancellation”, has been developed to remove in-band and correlated noises. The “Signal Separation” module embodies the new cyclic system reconfiguration method to separate body sounds. The “Pattern Recognition” module employs a stochastic pattern recognition algorithm that extracts key parameters for characterizing sound patterns with quantitative confidence levels. Then, the “Diagnosis” module identifies abnormal respiratory, cardiac, or other related conditions and diseases. Finally, the “Display and Storage” module provides a user interface for sound pattern feedback and display, information storage, and diagnostic outputs.

### Noise Reduction

The new noise reduction methodology of the present invention is uniquely designed to reduce the effect of signal/noise correlation. This method was derived on the basis of the unique nature of body sounds: (1) Breathing, heart, and upper airway sounds are not stationary, and usually have distinctive stages (inhale, exhale, and transitional pause in lung sounds, for example). (2) Sounds in signal-intensive stages, such as inhale and exhale stages in lung sounds, contain rich information about related body functions and can be processed for diagnosis. (3) During transitional pause, body sounds are very small and noises are dominant.

The noise canceling approach of this invention combines this unique method with the prior regular filtering techniques. The new method first uses a band-pass filter to eliminate the off-band noises (for example, sensors rubbing with skin or chest movement,). After-filtering signals are then used in conducting channel identification during the pause interval, and noise cancellation during the signal-intensive stages. Upon establishing a reliable model of noise transmission channels, noise cancellation can be achieved even when signal and noise are highly correlated during inhale and exhale. Therefore, the method introduced in this invention complements the traditional filtering and ANC for applications in which time-varying statistical features render ANC ineffective, leading to significantly improved quality of noise cancellation.

The method of time-shared adaptive noise cancellation has been shown to reduce the impact of inherent noises on accuracy of sound pattern recognition [20,20,21], The method of the present invention utilizes the unique features of lung sounds, heart sounds, snoring, and other body sounds. By combining cyclically reconfigured system identification for channel modeling, frequency-domain filtering, stochastic noise separation, the present method provides a far more robust and effective noise reduction than what was included in prior patents. Prior method patents proposed use of signal magnitudes and slopes to separate noise and signals. It is well known that such separations are not applicable to most noise cancellation cases. The noise cancellation method of the present invention includes the following new features:

1. A virtual noise representation by placing noise reference sensors at strategically selected locations. These locations have two key requirements: (1) They do not receive too much lung, heart or snoring sounds. (2) They are relatively close to signal sensors for lung, heart, or snoring sounds. Typical locations include shoulders, arms, but are not limited to these.

Location proximity between the lung and reference sensors allows representation of noises from many sources to be approximated by a lumped noise near the reference sensor. The method replaces distributed noise sources (which are impossible to describe accurately and separately) with a lumped noise source.

2. Cyclic separation of phases in lung, heart, and snoring sounds. While the overall sounds of heart, lung and snoring are not stationary processes, signals that are confined in separate stages are approximately stationary. For example, for lung sounds, the phases are inhale, exhale, and pause. For heart sounds, the phases are systolic, and pause. Mathematically, if all inhale segments of a breathing sound are extracted and concatenated into a single waveform, then this waveform is approximately stationary. This formulation allows this invention to apply powerful modeling and signal processing methodologies that are applicable only to stationary processes.

3. Time-shared noise cancellation. It is observed that due to diminishing lung sounds during the pause interval, the correlation between the sound and noise in the pause interval is much smaller than that for inhale and exhale processes, leading to our time-shared adaptive noise cancellation algorithm. The measured lung sound during the pause stage is essentially the output of the noise channel in that interval. As a result, we can use input/output pair to identify the noise transmission channel in this interval. This will not require any assumption regarding independence of signals and noises. The key steps in the algorithm are:

(1) During a pause stage, the measured noise reference (virtual input) and lung sound (output) are used to identify the noise channel.

(2) During the inhale and exhale phases, the estimated noise channel model is used to extract the original lung sound.

4. Recursive algorithms for channel identification. Adaptive filtering and stochastic approximation algorithms are used to derive recursive algorithms to update noise channel models and to achieve noise cancellation, from cycle to cycle. This cycle-to-cycle recursion is computationally very efficient since models are updated by using only new measurements and no past data needs to be stored or remembered. Also, by gradually discarding old data via, for example, exponential discarding data windows, this method can in fact track time-varying channel characteristics, that can be used in continuous monitoring and diagnosis of breath sounds.

5. Enhanced method of noise cancellation by combining time-shared adaptive noise cancellation with filtering and stochastic separation. The time-shared noise cancellation is further enhanced by targeted filtering and stochastic separation.

6. Individually targeted frequency filtering. The novelty of this feature of the invention is to identify an individual patient's baseline frequency ranges for targeted diagnosis conditions (such as “normal” and “crackle”) from initial data. These frequency ranges are then used to generate an individualized frequency filter that separates signals outside these frequency ranges since they are irrelevant to diagnosis targets.

### Signal Separation

Signal separation involves two source signals s1 and s2. For example, in heart/lung sound separation problems, s1 is the heart sound and s2 is the lung sound. The measurements x1 and x2 are subject to cross interference from both source signals. A typical example in medical applications is separation of heart and lung sounds. In this case, the original source signals are heart and lung sounds. Their measurements, either by using stethoscopes or acoustic sensors, are subject to signal interference in which both heart and lung sounds are heard in each measured signal. The signal transmission channels are unknown. The goal is to generate authentic source signals s1 and s2 by using only the measurements x1 and x2. Since the channel transfer functions are unknown and may vary with time and/or operating conditions, they must be identified in real time. As a result, separation of heart from lung sounds becomes a problem of adaptive signal separation.

One key feature used in this invention for signal separation is the cyclic nature of these two signals: Each signal undergoes phases: signal emerging (inhale and exhale for lung sounds and heart beating for heart sound) and pausing (lung sound pausing in between inhale and exhale and heart sound pausing in between heart beats). This invention discloses how these vital sign features can be used effectively in separating the signals.

The main approach of cyclic system reconfiguration is explained as follows. The 2×2 system has two signal sources s1 and s2 and two observations x1 and x2. The observations are assumed to be convolution sums of the source signals, with unknown source-to-observation channels G12 (interference of sound 2 by sound 1) and G21 (interference of sound 1 by sound 2). The signal interference occurs when each observation contains signals from both sources. The signals from each source before interference from the other source are called p1 and p2, which are the authentic sounds that can be heard during auscultation without interference. The methodology of this invention is designed to recover p1 and p2. It is understood by those versed in the art that if all transmission channels are known, p1 and p2 can be directly recovered by mathematical inversion of the 2×2 system.

But the signal transmission channels G12 and G21 are unknown. As a result, obtaining p1 and p2 is a blind signal separation (BSS) problem. There exist many approaches to the BSS problem such as output de-correlation, higher order statistics, neural network based methods, minimum mutual information and maximum entropy, and geometric based methods. Although the underlying principles and approaches of those standard methods are different, most of these algorithms assume that the original signals are statistically independent and the separation processes are then dependent on this key property. The present invention introduces a new method to identify the unknown transmission channels by simplifying the complex BSS problem to a set of regular identification problems without any constraints on the independence of the source signals.

The new method of this invention requires that the source signals should have some rhythms, namely the signals undergo intervals of existence and almost non-existence sequentially and yet are non-synchronized. Many biomedical signals bear these features, including for example heart beats, lung sound, and snoring. The approach of this invention uses these features to reconfigure iteratively the transmission channels so that the blind identification problem can be reduced into a number of regular identification problems.

The following intervals are consequently recognized by the invention.

(1) Interval Class I: p1 is nearly zero and p2 is large.

In this case, x1=G21*p2 and x2=p2. As a result, sensor measurements x1 and x2 during Interval Class I can be used to identify the transmission channel G21.

(2) Interval Class II: p2 is nearly zero and p1 is large.

In this case, x2=G12*p1 and x1=p1. As a result, sensor measurements x1 and x2 during Interval Class II can be used to identify the transmission channel G12.

Once the transmission channels have been identified, this invention can get the desired separated signals p1 and p2 by inverting the transmission system.

### Sound Pattern Recognition and Diagnosis

The new pattern recognition methodology of the present invention discloses a new technique of individualized pattern recognition and diagnosis [20,26]. The key properties of pattern recognition accuracy, confidence levels, noise impact, and noise reduction are rigorously established. The invention starts with a set of characterizing variables that can be extracted from sound waveforms. For an example of lung sounds, these variables may include, but are not limited to, inhale length and strength, exhale length and strength, breath cycle length, in the time domain; and center frequency, power, frequency bandwidth, for inhale and exhale individually, in the frequency domain. Changes in these variables provide information to the invention algorithm for determination of lung sound pattern variations. The goals of sound pattern recognition and diagnosis in this invention include: (1) to dynamically capture changes in these key parameters; (2) to relate these changes to potential causes. The invention includes the following improvements over prior pattern recognition methods:

1. A general methodology to extract multiple sound parameters that can be used to characterize different sound patterns, depending on targeted applications. These parameters include, as an example, both inhale and exhale, time-domain and frequency-domain characteristics. Typical parameters consist of an inhale parameter vector of time interval, power, magnitude, frequency center, frequency band, etc., and a similar vector for exhale. Although the above variables have been used in their individual applications as useful characteristics of lung sounds, a general methodology of multi-variable analysis is new. The new methodology is general and applicable if other parameters are used.

2. Individualized parameter distributions that are derived from data using stochastic analysis methods. It is well known that patient sound patterns vary dramatically and population patterns are not a good approach for diagnosis. This invention makes it possible to define individual baselines for diagnosis.

3. A dynamic sound pattern tracking method that captures pattern shifting in each patient. The main issue for sound pattern classification is to dynamically capture the changes of the individualized patient key parameters. To detect sound pattern shifting (or example deviation from normal ventilated lung sounds towards wheezing), this invention treats these calculated parameters, over each breath cycle, as stochastic processes. A method of windowed averaging with gradual data discarding is used to track pattern changes in a patient.

4. A method of optimally selecting diagnosis regions to maximize accuracy of diagnosis. The method is based on a stochastic optimization procedure that uses a multi-objective performance index to minimize combined errors of “misdiagnosis” and “false alarm.” The invention method generates diagnosis regions accurately, individually, and objectively. This is in contrast to prior methods, that use subjectively selected thresholds, which depend on “population average values,” or trial-and-error decision processes.

5. A recursive decision process that is computationally efficient for continuously monitoring lung sounds. This invention includes a recursive method which updates diagnosis regions when new data have been acquired. Consequently, the method of the invention does not need to compute the regions repeatedly when observation of lung sounds produces new parameters continuously over a long period of time.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 shows a general overview of the function modules of the system and method with an embodiment of the present invention.

FIG. 2 shows the prior art of traditional stethoscope technology and its main function modules.

FIG. 3 shows the prior art of analog electronic stethoscope technology and its main function modules.

FIG. 4 shows the prior art of digital electronic stethoscope technology and its main function modules.

FIG. 5 shows the prior art of combined stethoscope technology and portable devices and their main function modules.

FIG. 6 shows the prior art of multi-sensor sound analysis systems and their main function modules.

FIG. 7 shows a general block diagram overview of the system and method including a multiple-sensor array in accordance with an embodiment of the present invention.

FIG. 8 shows the modules and their connections to empirical devices of the system and method in accordance with an embodiment of the present invention.

FIG. 9 shows a block diagram of the overall system structure, configurations and function modules in accordance with the present invention.

FIG. 10 shows a block diagram showing signal interference and noise corruption in body sound transmission channels in accordance with an embodiment of the present invention.

FIG. 11 shows the simplified channel configurations for signal separation and noise cancellation in accordance with an embodiment of the present invention.

FIG. 12 shows the main system reconfiguration method that identifies signal transmission channels iteratively, separates body sounds, and removes noises in accordance with the present invention.

FIG. 13 shows a block diagram for the method of representation of distributed noise sources with a lumped noise source near the reference sensor in accordance with an embodiment of the present invention.

FIG. 14 shows a block diagram for the representation method used in the time-shared and individualized noise cancellation module in accordance with an embodiment of the present invention.

FIG. 15 shows a block diagram for the real-time and individualized adaptive pattern extraction module in accordance with an embodiment of the present invention.

FIG. 16 shows a block diagram for the optimized dynamic diagnosis module in accordance with an embodiment of the present invention.

FIG. 17 shows a typical respiratory sound where for signal processing with an embodiment of the present invention.

FIG. 18 shows a diagram showing a comparison in the time domain of results for noise cancellation using ANC and using the method of Time-Shared ANC in accordance with an embodiment of the present invention.

FIG. 19 shows a diagram illustrating the impact of noise on lung sound patterns.

FIG. 20 shows a diagram comparing results in the time domain for noise cancellation using ANC versus using the method of Time-Shared ANC in accordance with an embodiment of the present invention.

FIG. 21 shows is a diagram showing a comparison in the frequency domain of results for noise cancellation using ANC versus using the method of Time-Shared ANC in accordance with an embodiment of the present invention.

FIG. 22 shows a diagram showing characteristics for normal and abnormal lung sounds in accordance with an embodiment of the present invention.

FIG. 23 is a diagram showing a histograms of sample points of sound parameters giving a quantitative analysis on parameter vector distributions in accordance with an embodiment of the present invention.

FIG. 24 is a diagram comparing simulations were performed on identification errors of the recursive least-squares algorithm.

FIG. 25 is a diagram showing parameter data points on normal sound and wheeze in accordance with an embodiment of the present invention.

FIG. 26 is a diagram showing noise impact on normal sound and wheeze in accordance with an embodiment of the present invention.

FIG. 27 is a diagram showing confidence regions for pattern recognition in accordance with an embodiment of the present invention.

FIG. 28 is a diagram showing mean trajectories of parameters without noise cancellation

FIG. 29 is a diagram showing pattern recognition after noise reduction by time-shared adaptive noise cancellation in accordance with an embodiment of the present invention.

FIG. 30 is a diagram showing measured heart and lung sounds and the signals after off-band noise filtering.

FIG. 31 is a diagram showing measured heart and lung sounds and the signals after noise cancellation and signal separation by using the cyclic system reconfiguration method of this invention.

### DETAILED DESCRIPTION OF THE PREFERRED EMBODIMENTS

The following description of the preferred embodiment(s) is merely exemplary in nature and is in no way intended to limit the invention, its application, or uses.

### Hardware Overview of the Preferred Embodiment

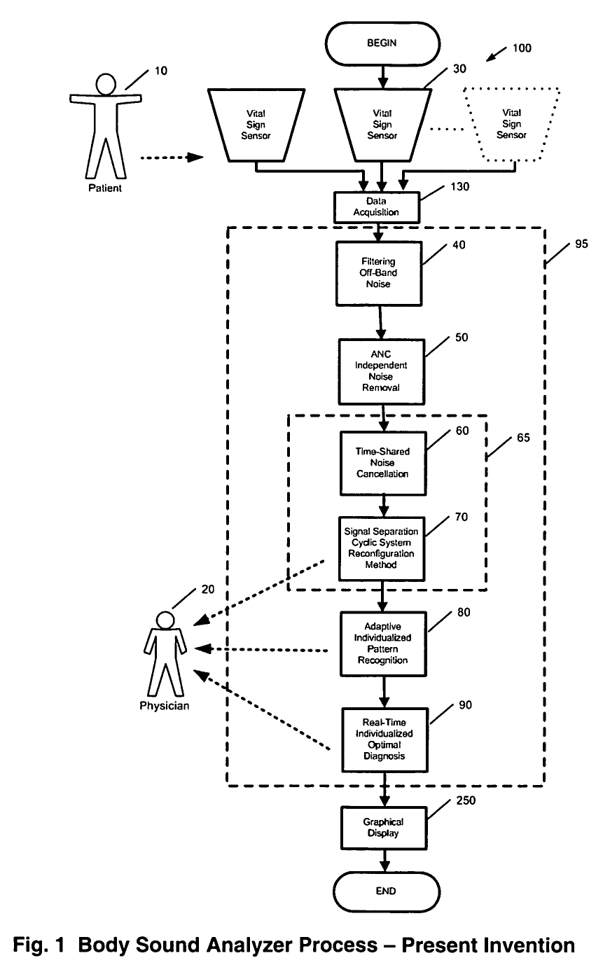

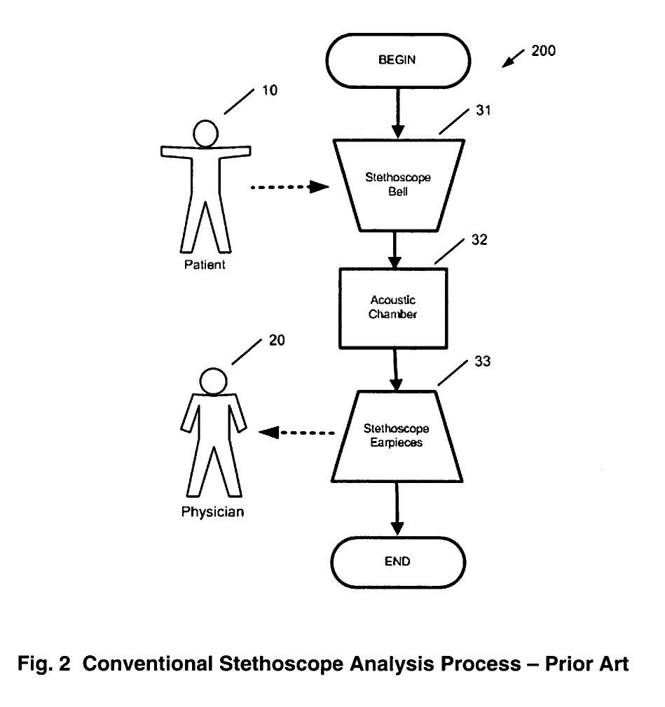

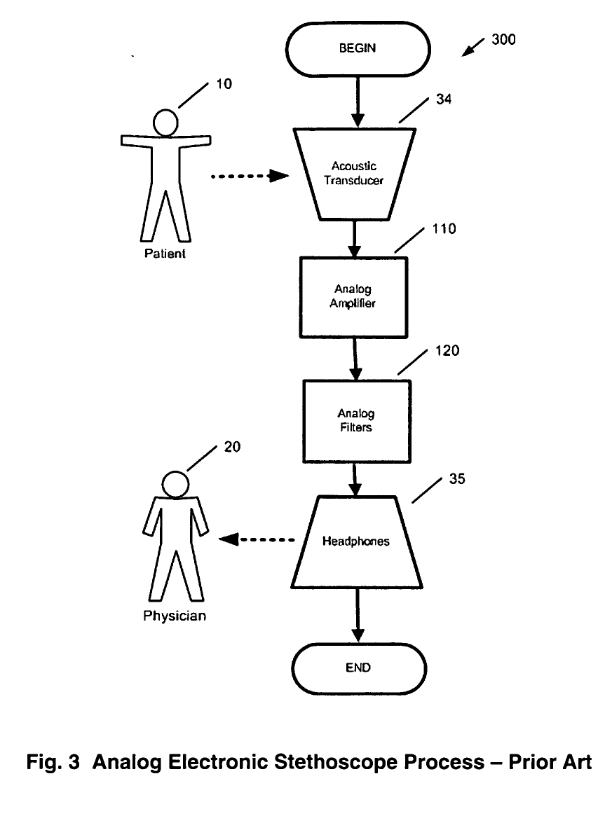

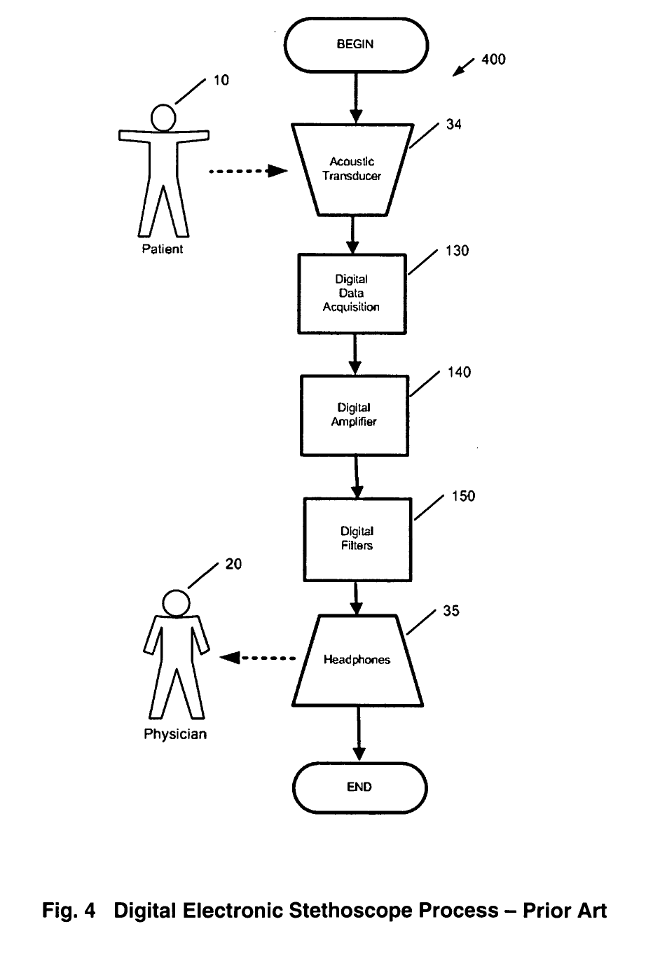

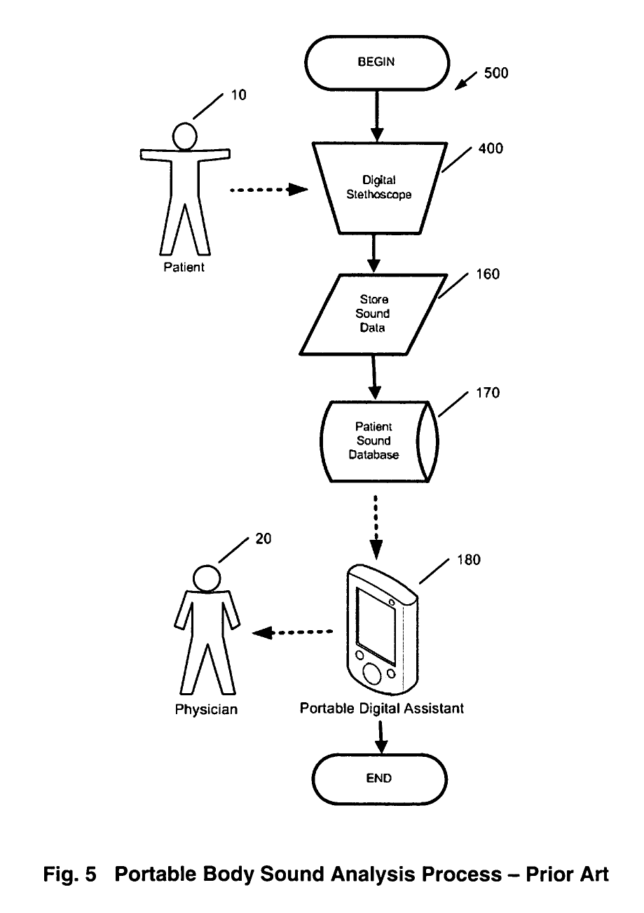

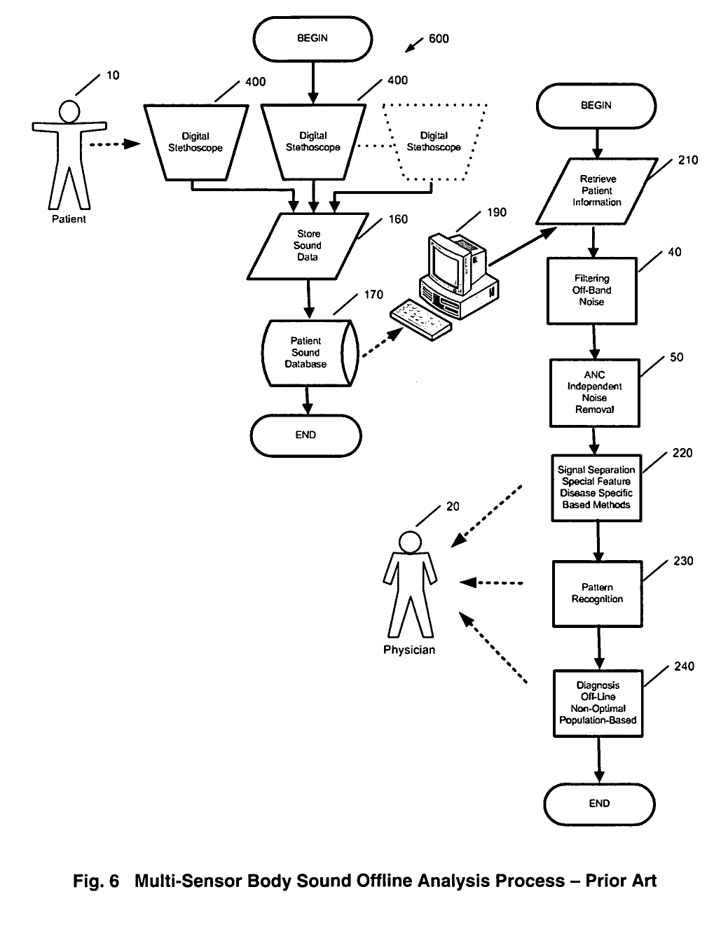

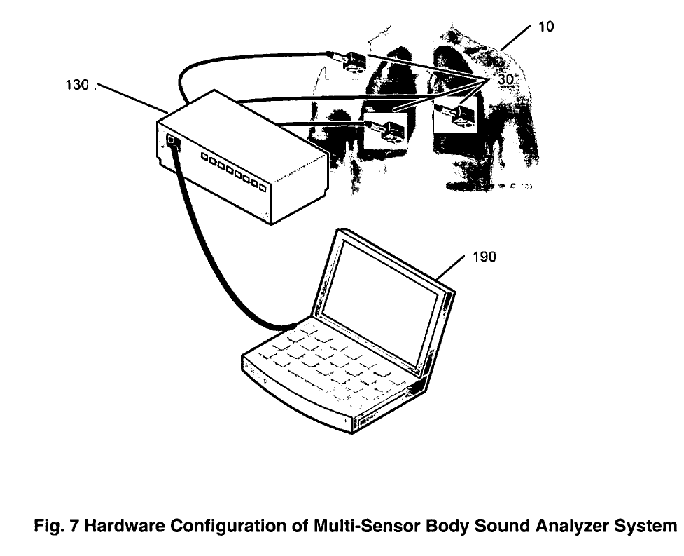

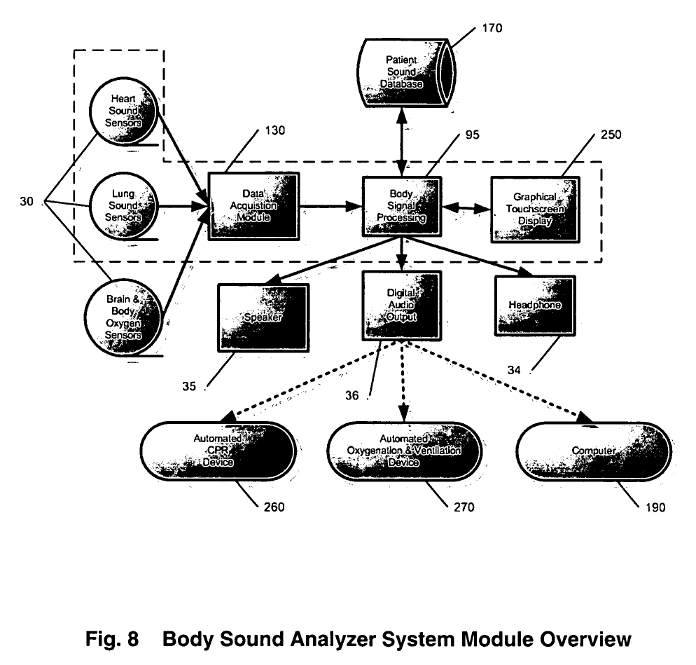

FIG. 1 shows an overview of the Body Sound Analyzer System 100 modules and processes including inputs and output devices. The invention includes a sound acquisition module which consists of several vital sign sensors 30 for measuring body sounds continuously which have their signals acquired by a data acquisition module 130, that is connected to a computer 190. Data acquisition module 130 and computer 190 may be embedded in a single Multi-Sensor Body Sound Analyzer System as shown in FIG. 7. The sensors 30 can be any type of acoustic sensors that are sufficiently sensitive and have satisfactory signal/noise ratios. Typical acoustic sensors include, but not limited to, special microphones, electronic stethoscopes, small accelerometers, and special-purpose body sound sensors. As shown in FIG. 1 the sensors will be placed on auscultation sites on a patient 10 for targeted body sounds, such as tracheal, bronchial, heart, etc., and for noise references. Sound waves acquired by the sensors will then be processed using the Body Sound Signal Processing System 95. In order to obtain noise measurements that represent lumped impact of distributed and multi-source noises on the lung sensors, noise reference sensors are placed on the patient 10 in the vicinity of the sound sensors 30. Sound waves acquired by the sensors 30 are then fed into an analog/digital data acquisition module 130 for signal input, scaling, sampling rate synchronization, and other signal conditioning. The data acquisition module 130 is then connected to a computer 190 which implements the Body Sound Signal Processing System 95. As shown in FIG. 7, the systems of 130 and 190 may be embedded into one hardware unit. Shown in FIG. 1, sound signals and noise references are then inputted to the following consecutive function modules 95: a filter module for removing off-band noise 40, an adaptive noise cancellation module to remove independent noise 50, a noise cancellation and signal separation module 65 for removing in-band and other noises and separating sound signals to overcome signal interference, a pattern recognition module 80 and diagnosis module 90 for diagnosis. The processed sound signals and parameters are then sent to a display and storage module 250 for audio replay or graphical display, as well as data storage for future utility. Shown in FIG. 8 is an overview of the hardware modules that includes the Body Sound Signal Processing System 95 associated input and output devices and hardware. As shown in FIG. 8, the sensors 30 may include sensors measuring lung sounds, heart sounds and even brain and body oxygen sensors. The invention can perform its signal processing on any suitable signals. The physician 20 can be apprised of the invention processing results via speaker 35, digital audio output 36, or headphones 34. The system of the invention can become an integral device in healthcare operations by interface with among others automated CPR devices 260, automated oxygenation and ventilation devices 270 or other computing devices 190.

### Software Overview of the Preferred Embodiment

As shown in FIG. 7, the signals are first conditioned and synchronized by the data acquisition module 130. To obtain authentic lung sounds, signals are filtered to remove off-band 40 and independent noises 50. The time-shared noise cancellation module 60 and signal separation using cyclic system reconfiguration method module 70 embody the methodology for cyclic system reconfiguration and adaptive channel identification for removing in-band and correlated noises 65. The adaptive individualized pattern recognition module 80 employs a stochastic pattern recognition algorithm that extracts key parameters for characterizing sound patterns with quantitative confidence levels. Then, the real-time individualized optimal diagnosis module 90 identifies abnormal respiratory conditions and diseases. Finally, the graphical display 250 and storage modules 170 provide a user interface for sound pattern feedback and display, information storage, and output of diagnoses. Also shown in FIG. 7 are several lung sound sensors 30 (that can be special microphones, accelerometers, electronic stethoscopes, or specially-designed MEMS acoustic sensors) on auscultation sites such as tracheal and bronchial, and one or more noise reference sensors.

### Combined Signal Separation and Noise Cancellation Module

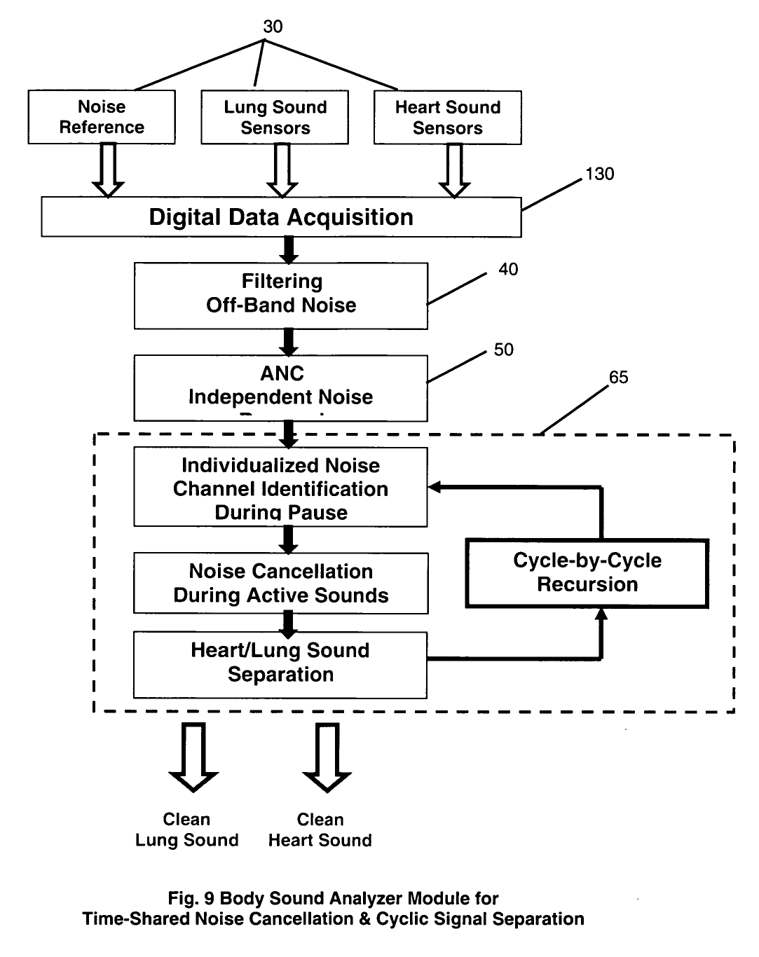

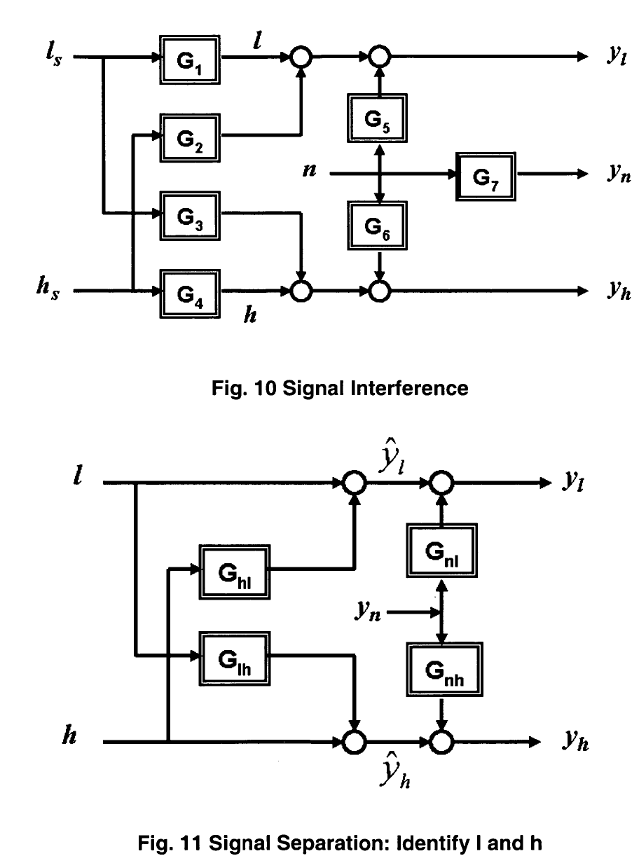

FIG. 1 65 is a combined signal separation and noise cancellation module. FIG. 9 65 show a block diagram of the model structure, configurations and including function modules for signal separation and noise removal. When two or more body sounds must be measured simultaneously, their transmission channels are typically those shown in FIG. 10, in which both signal interference and noise corruption in body sound transmission channels are present. To obtain authentic body sounds, the transmission channels are simplified to those shown in FIG. 11.

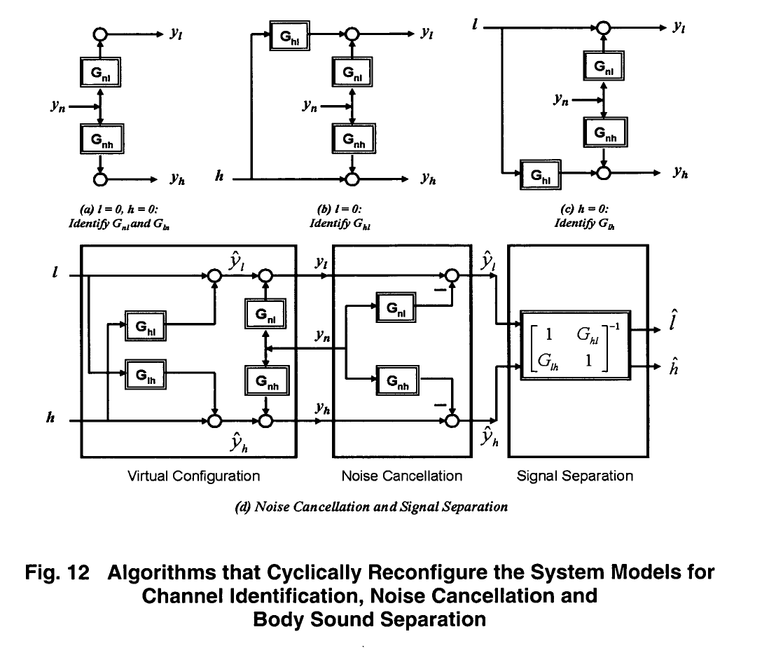

FIG. 12 shows the diagram for the main system reconfiguration method that identifies signal transmission channels iteratively, separates body sounds, and removes noises. Using the heart and lung sound separation as an example, FIG. 12(a) shows that when both heart and lung sound are near zero, the sensor measurements are used to identify noise transmission channels. When the lung sound is near zero, the system removes noise and then identify the heart-to-lung interference channel Ghl in FIG. 12(b). Similarly, when the heart sound is near zero, the system removes noise and identify the lung-to-heart interference channel Glh in FIG. 12(c). Once all transmissions are identified, FIG. 12(d) shows that the system first removes noises and then separate heart and lung sound by inverting the transmission system. This framework is general and can be used for other body sounds as well.

### Time-Shared and Individualized Noise Cancellation Module

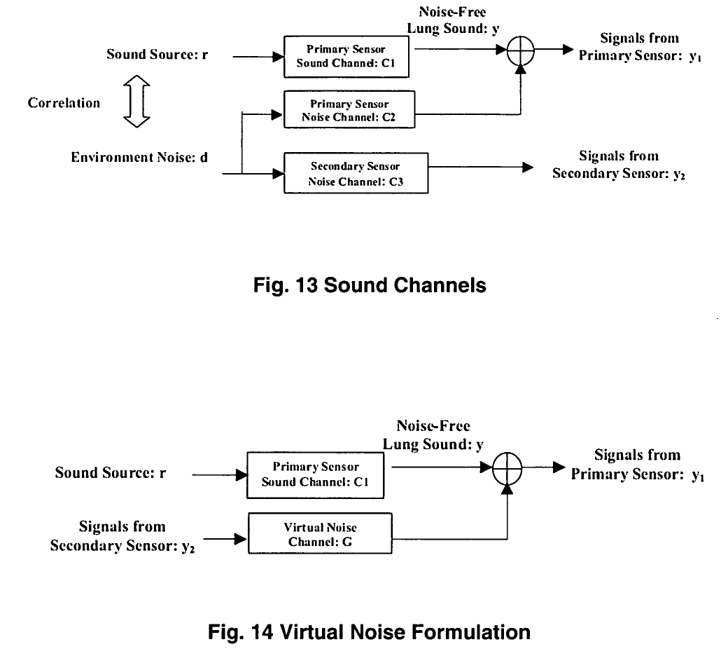

When only one body sound must be extracted from noise-corrupted measurements, this function module FIG. 1 60 is in effect. FIG. 13 shows the block diagram of the method incorporated in this module of the invention for representation of distributed noise sources with a lumped noise source near the reference sensor. This module treats the measurement from the reference sensor as a virtual noise source in which the distributed noise sources are replaced by a lumped noise source y2, as shown in FIG. 14. Then the problem of noise cancellation is reduced to identification of the virtual noise channel and the noise free target signal can be approximately extracted.

### Adaptive Individualized Pattern Extraction Module

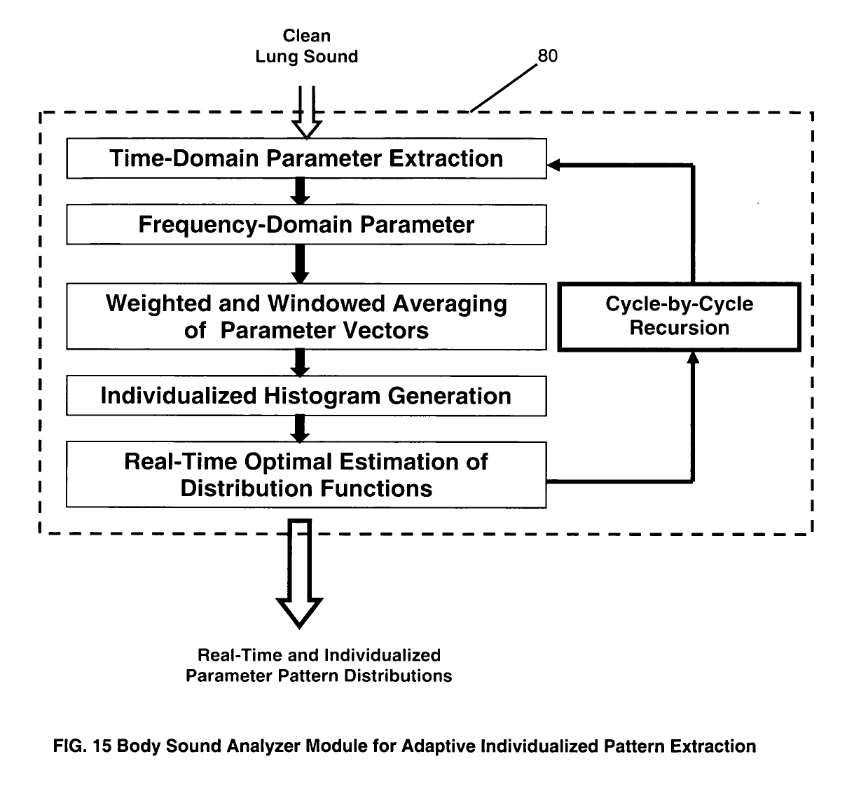

This module is shown in FIG. 1 80. The function blocks of this module are shown in FIG. 15. The key parameters in both the time domain and frequency domain are first extracted. The parameters are time sequences. They are averaged over a moving window to reduce randomness. Then individualized histograms are generated to capture their statistical properties. The histograms serve as data points to generate in real-time parameter distribution functions that are unique to a patient.

### Real-Time Individualized Optimal Diagnosis Module

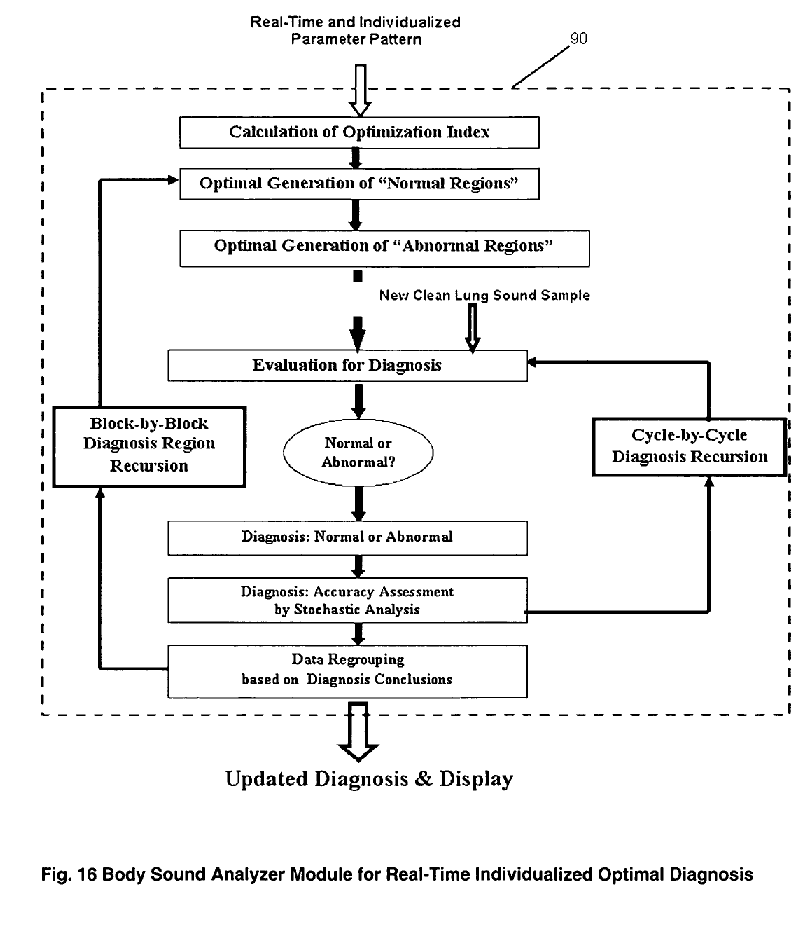

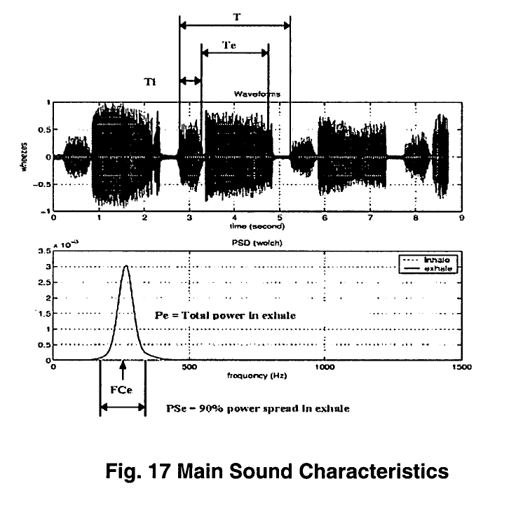

This module is shown in FIG. 1 90. The function blocks of this module are shown in FIG. 16. Diagnosis is performed in real-time. Diagnosis regions are generated recursively, by incorporating information from new parameter values of sound samples. The diagnosis regions are used to decide if an abnormal sound sample has been found. The decision is based on an optimal decision strategy that minimizes decision errors. Then the diagnosis regions are updated by the new data.

FIG. 17 is a typical respiratory sound where for signal processing with an embodiment of the present invention, a ventilation or breathing cycle is divided into three stages: Inhale (Ti), exhale (Te), and transitional pause (T-Ti-Te).

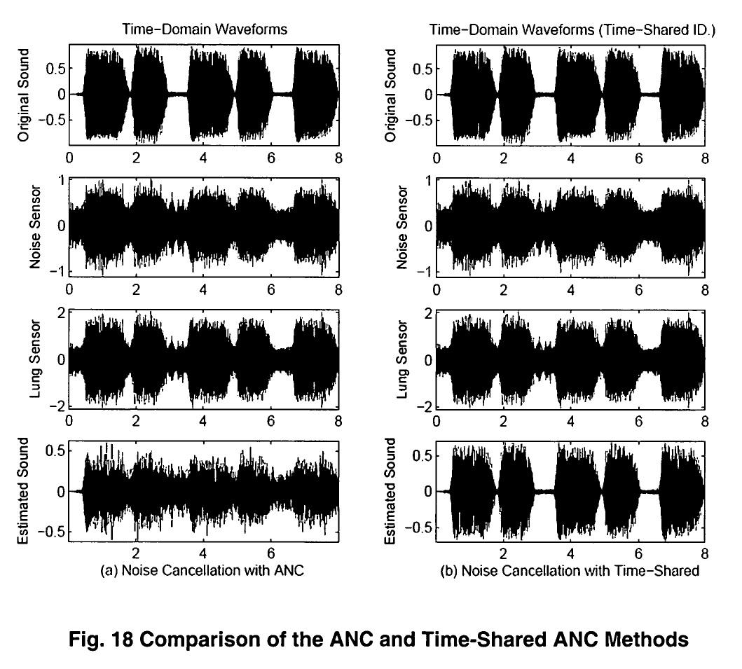

FIG. 18 is a diagram showing a Time Domain Comparison of results for noise cancellation using ANC and using the method of Time-Shared ANC that shows deterioration of noise cancellation efficiency in lung sound analysis when correlations exist in accordance with an embodiment of the present invention.

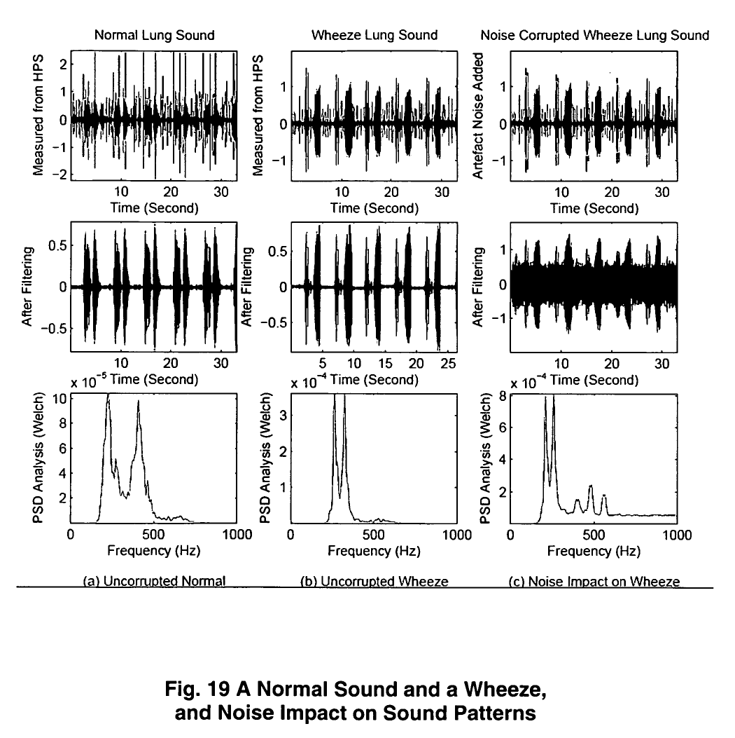

FIG. 19 is a diagram of showing an illustration of noise impact on lung sound patterns. FIG. 19(a) is a typical normal breathing sound and FIG. 19(b) an expirational wheeze. The top figures in FIG. 19 are the raw data. Due to low-frequency noises from sensor contact surfaces, the breathing patterns are not obvious. A high-pass filter is used to eliminate the noise under 200 Hz. After filtering, the difference between normal and wheeze lung sounds can be clearly seen from their time domain waveforms. In frequency domain analysis, the wheeze can be further characterized by a substantial narrowing of the spectrum, shifting of the center frequency (towards low pitch in this example), etc. For this example, sounds are obviously very clean with minimum noise corruption. Sound patterns are significantly altered when noise artifacts are present. FIG. 19(c) shows the corrupted wheeze signal, both in its time-domain waveform and frequency-domain spectrum. It is apparent that in a noisy environment, the time-domain waveforms of a wheeze are distorted to the point that it is no longer possible to recognize sound patterns in accordance with an embodiment of the present invention.

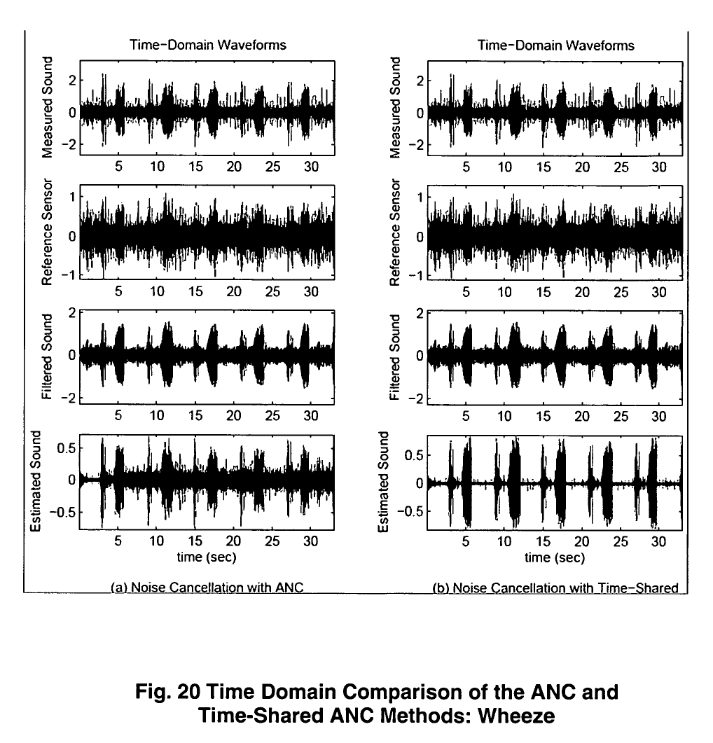

FIG. 20 is a diagram showing a Time Domain Comparison of results for noise cancellation using ANC versus using the method of Time-Shared ANC on Wheeze sounds in accordance with an embodiment of the present invention.

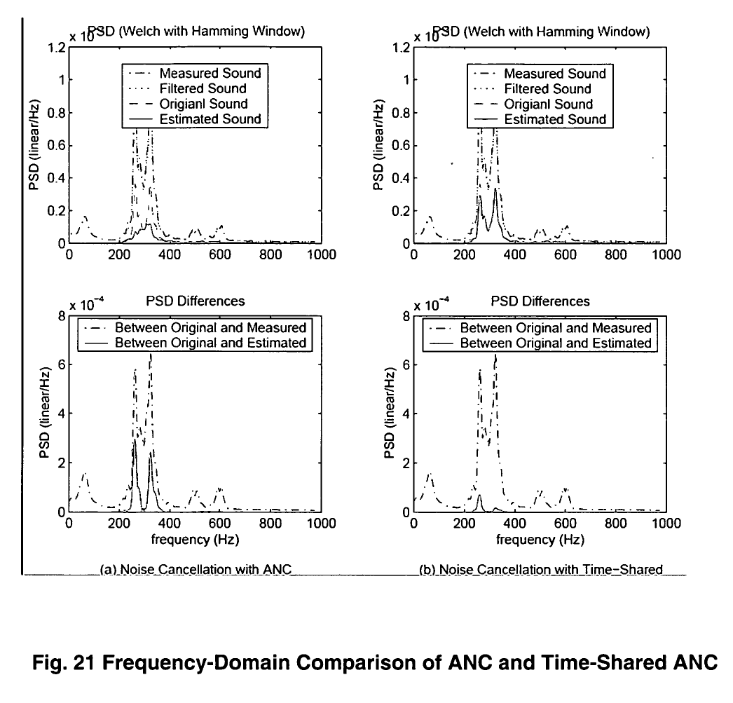

FIG. 21 is a diagram showing a Frequency Domain Comparison of results for noise cancellation using ANC versus using the method of Time-Shared ANC in accordance with an embodiment of the present invention. The noise spectrum overlaps with the lung sound spectrum. The estimated lung sound restores the power spectrum of the original lung sound. The results for ANC compare the spectra of the measured lung sound, estimated lung sound and original lung sound (the top plot of FIG. 21(a)). ANC can only reduce noises that are not correlated with the lung sound in spectra, as shown in the bottom plot of FIG. 21(a). Time-shared ANC provides a more effective noise reduction in spectra, as shown in FIG. 21(b). It can cancel most noises no matter if they are correlated with lung sounds or not.

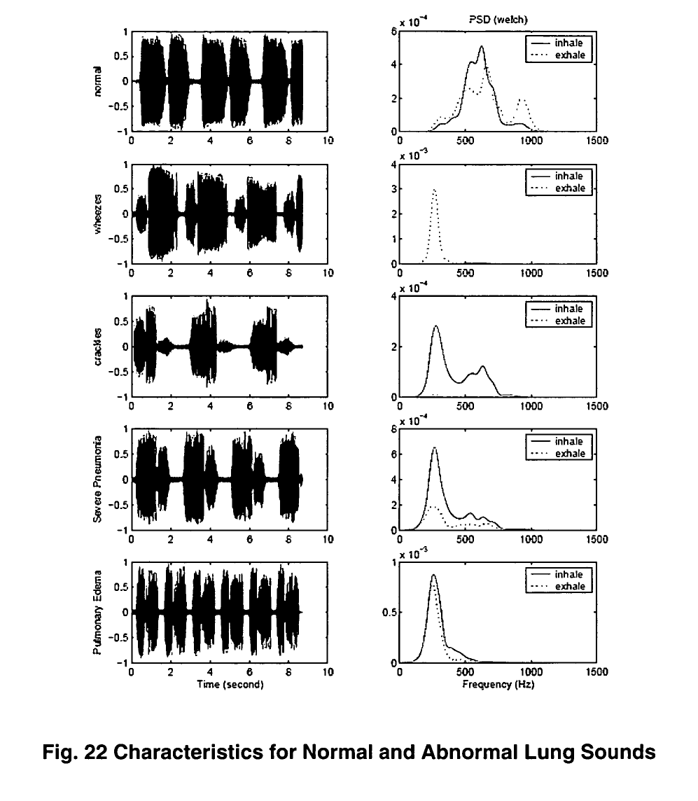

FIG. 22 is a diagram showing Characteristics for Normal and Abnormal Lung Sounds. To understand what variables might be useful to capture pattern changes in lung sounds, we illustrate some typical normal and abnormal lung sound waveforms and their frequency spectra during inhale and exhale in FIG. 22. For example, the wheeze can be clearly characterized by a substantial narrowing of spectrum, shifting of center frequency (towards low pitch in this example), and power imbalance between inspiration and expiration. in accordance with an embodiment of the present invention.

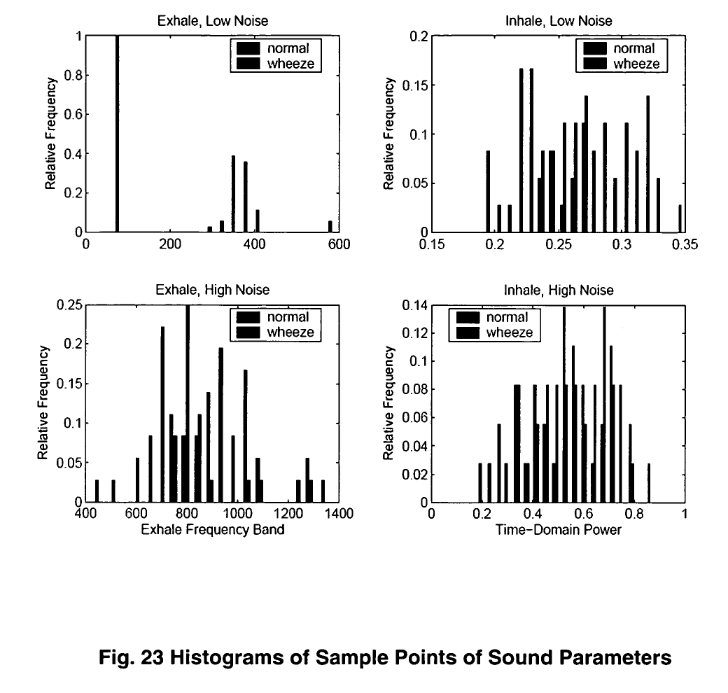

FIG. 23 is a diagram showing a Histograms of Sample Points of Sound Parameters giving a quantitative analysis on parameter vector distributions. It is noted that when noise level increases sound patterns have larger deviations and have a pattern shifting as well. As discussed before, inherent noises result in pattern shifting which cannot be eliminated by stochastic averaging. Reduction of impact from inherent noises must be done by noise cancellation techniques, which will be discussed later. On the other hand, increased sensor noises result in larger deviations. Averaging can be used when the size of data samples becomes larger.

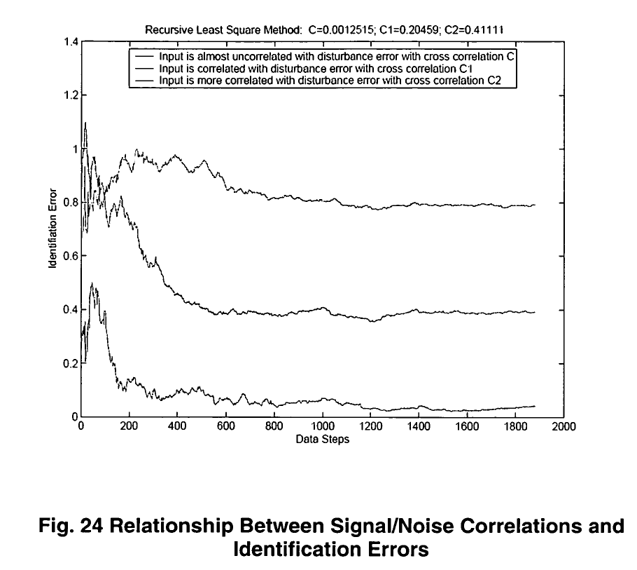

FIG. 24 is a diagram showing simulations were performed on identification errors of the recursive least-squares algorithm. Three cases were compared: (1) the input signal u(k) is uncorrelated with the disturbance signal d(k); (2) u(k) is correlated with d(k) of a moderate level; (3) the correlation between u(k) and d(k) is more severe than the second case. FIG. 24 illustrates the trajectories of identification errors. The results clearly demonstrate that higher correlations between u(k) and d(k) lead to larger estimation errors and slower convergence rates. This simulation explains why our time-shared ANC method is more accurate and efficient in accordance with an embodiment of the present invention.

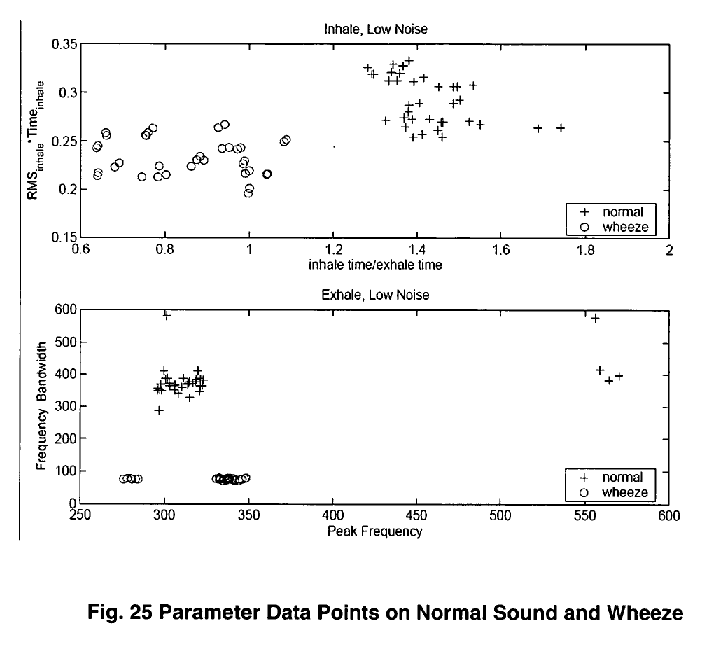

FIG. 25 is a diagram showing Parameter Data Points on Normal Sound and Wheeze in accordance with an embodiment of the present invention.

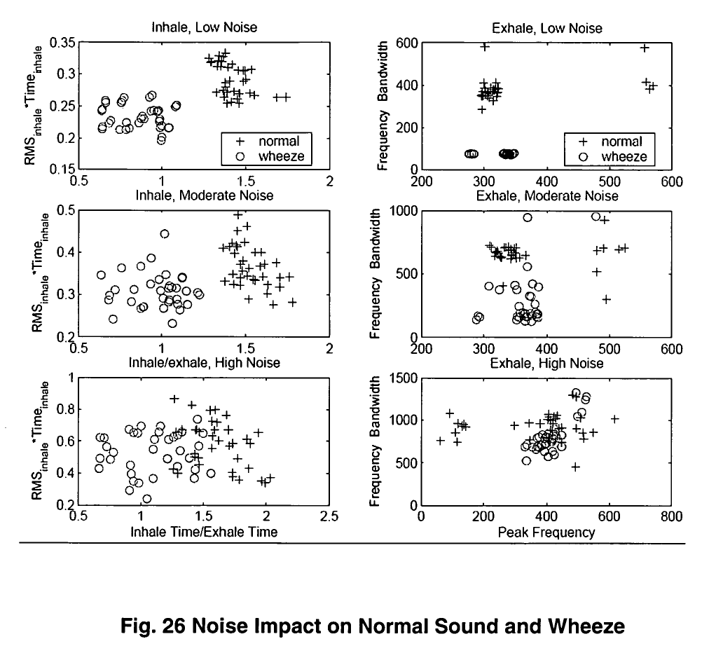

FIG. 26 is a diagram showing noise impact on normal sound and wheeze in accordance with an embodiment of the present invention.

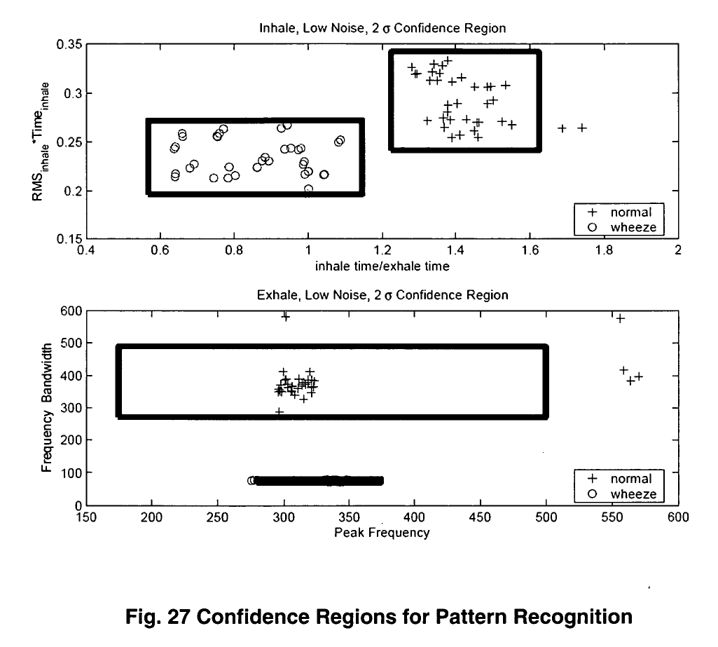

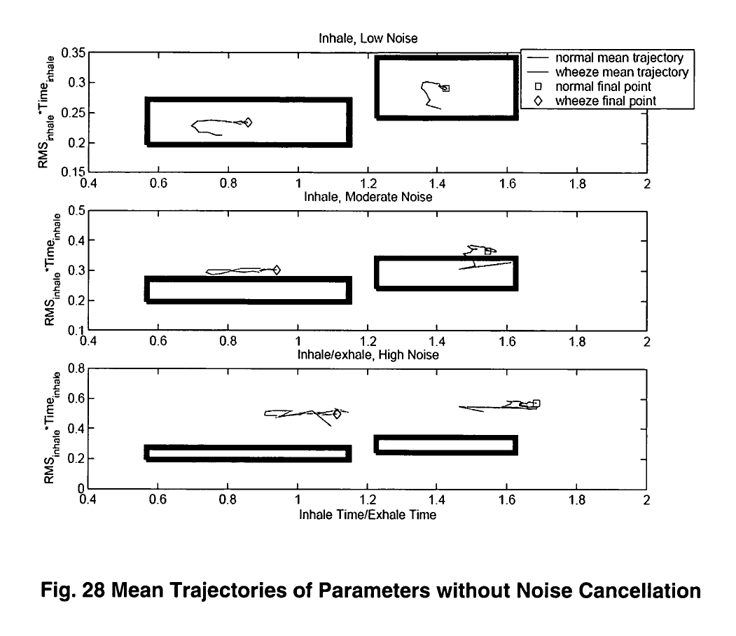

FIG. 27 is a diagram showing Confidence Regions for Pattern Recognition in accordance with an embodiment of the present invention.

FIG. 28 is a diagram showing Mean Trajectories of Parameters without Noise Cancellation in accordance with an embodiment of the present invention.

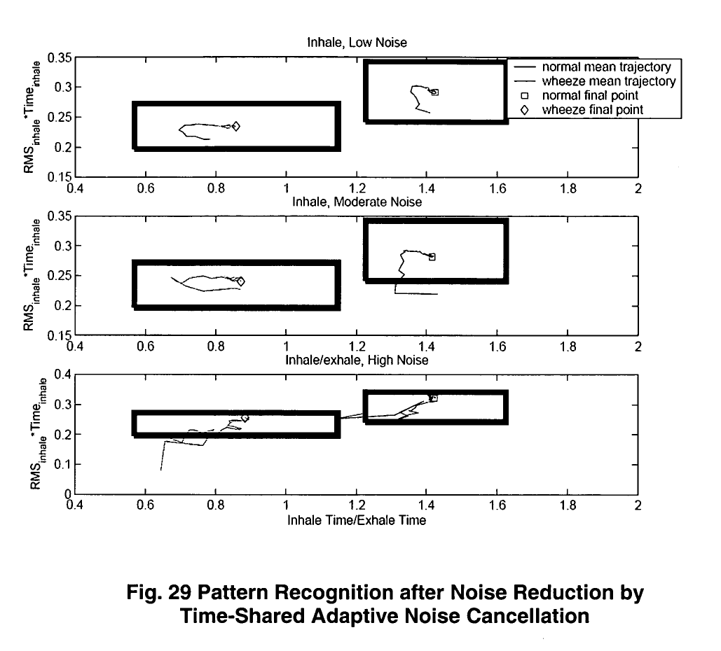

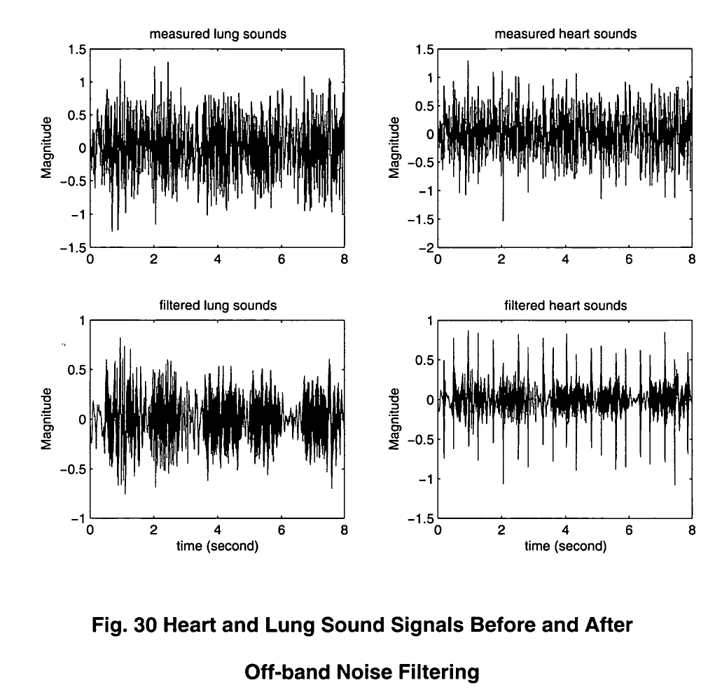

FIG. 29 is a diagram showing Pattern Recognition after Noise Reduction by Time-Shared Adaptive Noise Cancellation in accordance with an embodiment of the present invention.

FIG. 30 is a diagram showing measured heart and lung sounds and the signals after off-band noise filtering. It reveals that off-band noise removal is not sufficient to clarify these signals.

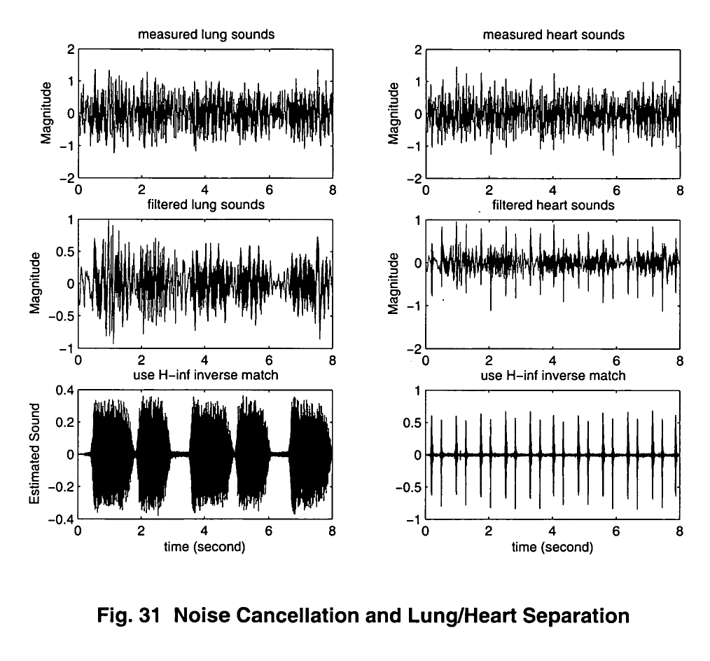

FIG. 31 is a diagram showing measured heart and lung sounds and the signals after noise cancellation and signal separation by using the cyclic system reconfiguration method of this invention. It reveals the effectiveness of the signal separation and noise cancellation method of this invention.

The description of the invention is merely exemplary in nature and, thus, variations that do not depart from the general design of the invention are intended to be within the scope of the invention. Such variations are not to be regarded as a departure from the intent and scope of the invention.

### REFERENCE KEYS IN FIGURES

10 Patient
20 Physician or Healthcare Worker
30 Vital Sign Sensor
31 Stethoscope Bell
32 Stethoscope Acoustic Chamber
33 Stethoscope Earpieces
34 Acoustic Transducer
35 Headphones
36 Digital Audio Output
40 Filtering of Off-Band Noise
50 Adaptive Noise Cancellation for Independent Noise Removal
60 Time-Shared Adaptive Noise Cancellation
65 Combined Cyclic System Reconfiguration Method for Signal Separation and Noise Cancellation
70 Cyclic System Reconfiguration Method for Signal Separation
80 Adaptive Individualized Pattern Recognition
90 Real-Time Individualized Optimal Diagnosis
95 Body Sound Signal Processing System
100 Body Sound Analyzer
110 Analog Amplifier
120 Analog Filters
130 Digital Data Acquisition
140 Digital Amplifier
150 Digital Filters
160 Process of Storing Sound Data
170 Patient Sound Database
180 Portable Digital Assistant
190 Computer System
200 Conventional Stethoscope System
210 Process of Retrieving Patient Information
220 Special Feature Based Signal Separation that is Disease Specific
230 Standard Pattern Recognition
240 Population-Based Diagnosis that is Off-Line Processed and Non-Optimal
250 Graphical Touch-Screen Display
260 Automated CPR Device
270 Automated Ventilation Device
300 Analog Electronic Stethoscope System
400 Digital Electronic Stethoscope System
500 Portable Body Sound Analysis System
600 Multi-Sensor Body Sound Off-Line Analysis System

### REFERENCES

[1] N. Gavriely, Y. Palti, and G. Alroy: “Spectral characteristics of normal breath sounds”, J. Appl. Physiol., vol. 50, no. 2, pp. 320-324, February 1981.
[2] V. Iyer, P. Ramamoorthy, H. Fan and Y. Plolysonsang: “Reduction of heart sounds from respiratory sounds by adaptive filtering”, IEEE Duns. Biomed. Eng., vol. 33, no. 12, pp. 1141-1148, 1986.
[3] L. J. Hadjileontiadis and S. M. Panas: “Adaptive reduction of heart sounds from lung sounds using fourth-order statistics”, IEEE fiuns. Biomed. Eng., vol. 44, no. 7, pp. 642-648, 1997.
[4] Z. Moussavi “An overview of heart-noise reduction of lung sound using wavelet transform based filter”, Proceedings of the 25^{th} Annual International Conference of the IEEE, pp. 458-461, September 2003.
[5] Barschdorff, D.; Bothe, A.; Rengshausen, U. , Heart sound analysis using neural and statistical classifiers: a comparison. Computers in Cardiology, 415-418, September 1989.
[6] Charleston, S.; Azimi-Sadjadi, M. R.; Gonzalez-Camarena, R., Interference cancellation in respiratory sounds via a multiresolution joint time-delay and signal-estimation scheme, Biomedical Engineering, IEEE Transactions, 1006-1019, October 1997.
[7] Guo Z. Durand L G. Lee H C. Allard L. Grenier M C. Stein P D. Artificial neural networks in computer-assisted classification of heart sounds in patients with porcine bioprosthetic valves. Medical & Biological Engineering & Computing. 32(3):311-6, 1994 May.
[8] Hadjileontiadis, L. J.; Panas, S. M. Adaptive reduction of heart sounds from lung sounds using fourth-order statistics. IEEE Transactions on Biomedical Engineering, 44 (7): 642-648, 1997.
[9] Hadjileontiadis, L. J.; Panas, S. M. Adaptive reduction of heart sounds from lung sounds using wavelet-based filter. Stud. Health Technol. Inform. 43 (Part B): 536-540, 1997.
[10] Haghighi-Mood, A.; Torry, J. N., Application of advanced signal processing techniques in analysis of heart sound. Signal Processing in Cardiography, IEE Colloquium, Aug. 1-5, 1995.
[11] Haghighi-Mood, A.; Torry, J. N. Time-varying filtering of the first and second heart sounds, 18th Annual International Conference of the IEEE, 950-951 vol. 3, November 1996.
[12] S. Haykin, Adaptive Filter Theory, Prentice-Hall, Englewood Cliffs, N.J., 1990.
[13] S. Haykin, ed. Unsupervised Adaptive Filtering, Vol. I and II, John Wiley & Sons, Inc., 2000.
[14] Kompis M. Russi E. Adaptive heart-noise reduction of lung sounds recorded by a single microphone. Proc. Annual International Conference of the IEEE, Vol. 2, 691-692, 1992.
[15] Longhini C. Portaluppi F. Arslan E. Pedrielli F. The fast Fourier transform in the analysis of the normal phonocardiogram. Japanese Heart Journal. 20(3):333-9, 1979 May.
[16] Lu Y S. Liu W H. Qin G X. Removal of the heart sound noise from the breath sound. Engineering in Medicine and Biology Society, 1988., Proceedings of the Annual International Conference of the IEEE, 175-176 vol. 1, November 1988.
[17] Pasterkamp H. Kraman S. Wodicka G. Respiratory sounds. Am. J. Respir Crit Care Med. Vol. 156: 974-987, 1997.
[18] B. Widrow and S. D. Stearns, Adaptive Signal Processing. Prentice-Hall, Englewood Cliffs, N.J., 1985.
[19] Hong Wang, Han Zheng, Le Yi Wang, Howard J. Normile, Jeremy Nofs, Adaptive Noise Cancellation and Lung/Heart Sounds Extraction Via Time-split Transmission Channel Reconfiguration, WSEAS Transactions on Biology and Biomedicine, Issue 3, Volume 3, pp. 204-211, March 2006.
[20] Razmig Haladjian, Hong Wang, Le Yi Wang, Han Zheng, “Computer-Aided Continuous Lung Sound Auscultation in Ventilated Patients”, Annual Conference of American Society of Anesthesiologists, Las Vegas, October 2004.
[21] H. Wang, L. Y. Wang, H. Zheng, R. Haladjian, M. Wallo, Lung sound/noise separation in anesthesia respiratory monitoring, WSEAS Transactions on Systems, Vol. 3, pp. 1839-1844, June 2004.
[22] Le Yi Wang, Hong Wang, Han Zheng, and George Yin, “Multi-Sensor Lung Sound Extraction Via Time-Shared Channel Identification and Adaptive Noise Cancellation”, 2004 IEEE Control and Decision Conference, December 2004.
[23] L. Y. Wang, G. Yin, H. Wang, Identification of Wiener Models with Anesthesia Applications, Int. J. of Pure and Applied Mathematical Sciences, pp. 35-61, 2004.
[24] Le Yi Wang and Hong Wang, Computers in Anesthesia, in Encyclopedia of Medical Devices & Instrumentation, 2nd Edition, Edited by Dr. John G. Webster, John Wiley \& Sons, Inc., February 2006.
[25] Hong Wang, Han Zheng, Le Yi Wang, Howard J. Normile, Jeremy Nofs, Separation of Lung and Heart Sound for Anesthesia Diagnosis, Proceedings of the 2006 WSEAS International Conference on Mathematical Biology and Ecology (MABE '06), Miami, Fla., USA, Jan. 18-20, 2006 (pp 63-68).
[26] H. Zheng, H. Wang, L. Y. Wang, and G. Yin, Time-Shared Channel Identification for Adaptive Noise Cancellation in Breath Sound Extraction, Journal of Control Theory and Applications, Vol. 2, No. 3, pp. 209-221, August 2004.
[27] Han Zheng, Hong Wang, Le Yi Wang, and George Yin, “Lung Sound Pattern Analysis for Anesthesia Monitoring”, 2005 American Control Conference, June 2005.
[28] Han Zheng, Le Yi Wang, Hong Wang, Cyclic System Reconfiguration for Adaptive Separation of Lung and Heart Sounds, 2006 ACC Conference, Minneapolis, Jun. 14-16, 2006.

### ATTACHMENTS

1 H. Wang, L. Y. Wang, H. Zheng, R. Haladjian, M. Wallo, Lung sound/noise separation in anesthesia respiratory monitoring, WSEAS Transactions on Systems, Vol. 3, pp. 1839-1844, June 2004.
2 Han Zheng, Hong Wang, Le Yi Wang, and George Yin, “Time-Shared Channel Identification for Adaptive Noise Cancellation in Breath Sound Extraction”, Journal of Control Theory and Applications, Vol. 2, No. 3, pp. 209-221, August 2004.
3 Le Yi Wang, Hong Wang, Han Zheng, and George Yin, “Multi-Sensor Lung Sound Extraction Via Time-Shared Channel Identification and Adaptive Noise Cancellation”, 2004 IEEE Control and Decision Conference, December 2004.
4 Han Zheng, Hong Wang, Le Yi Wang, and George Yin, “Lung Sound Pattern Analysis for Anesthesia Monitoring”, 2005 American Control Conference, June 2005.
5 Hong Wang, Han Zheng, Le Yi Wang, Howard J. Normile, Jeremy Nofs, “Separation of Lung and Heart Sound for Anesthesia Diagnosis”, Proceedings of the 2006 WSEAS International Conference on Mathematical Biology and Ecology (MABE '06), Miami, Fla., USA, Jan. 18-20, 2006 (pp 63-68).

What is claimed is:

1. A multi-sensor based and automated system for continuously and automatically reducing noise artifacts in signal measurements and separating target signals from their cross interferences, using time-shared and cyclic system reconfiguration methods. The system comprises:
a. multiple sensors which capture target signals and noises from a plurality of locations, Noise sensors are positioned such that the distributed background noises can be approximated as lumped noise sources;
b. a time-shared adaptive individualized noise cancellation module which reduces noise that may have overlapping frequency components with the target signals or is statistically correlated with the target signals;
c. a cyclic system reconfiguration module for target signal separation which uses the rhythmic but non-synchronized nature of the target signals to identify the sound transmission channels iteratively and perform separation of signals in real-time;
whereby target signals from multiple sources that have similar stochastic and frequency features are physically separated both from each other and also from extraneous sources of noise that may be statistically correlated with or have overlapping frequency components with the target signals.

2. A system for continuously and automatically performing pattern recognition and diagnosis of target signals during monitoring comprising:
a. a module for identifying and extracting multiple parameters to be used for characterization of said target signals;
b. a module for derivation of Individualized parameter distributions for said multiple parameters that are derived from data using stochastic analysis methods thereby defining individual baselines for diagnosis;
c. a dynamic pattern tracking module which dynamically captures the changes of said individualized key parameters by using stochastic processes;
d. an optimal diagnosis region selection module to maximize accuracy of diagnosis based for example on a stochastic optimization procedure that can use a multi-objective performance index to minimize combined errors thereby generating diagnosis regions accurately, individually, and objectively.
e. an electronic display which visually displays results of the pattern recognition information and the motion path of extracted key parameters;
whereby warning alarms for deviation of key parameters from their safe regions, diagnosis results, and remedial recommendations are output by said display.

3. The combined noise removal and signal separation system of claim 1 followed by the pattern recognition and diagnosis system of claim 2 wherein the combined overall system can automatically perform continuously noise reduction, target signal separation, pattern recognition, diagnosis, and display of diagnosis results in real-time.

4. The system of claim 1 wherein said sensors are acoustic and the target signals are body sounds, such as heart beats and lung sounds wherein the system generates clarified body sound attributes including among others the separation of heart from lung sounds.

5. The combined noise removal and signal separation method of claim 4 followed by the pattern recognition and diagnosis method of claim 2 wherein the overall system can automatically perform continuous monitoring and diagnosis of body conditions and diseases in real-time including for example separation of heart and lung sounds.

6. A multi-sensor based and automated method for continuously and automatically reducing noise artifacts in signal measurements and separating target signals from their cross interferences for any signals that have the features of rhythmic and non-synchronized cycles undergoing signal intensive and signal diminishing stages, using time-shared and cyclic system reconfiguration methods. The method comprises:
d. multiple sensors which capture target signals and noises from a plurality of locations, Noise sensors are positioned such that the distributed background noises can be approximated as lumped noise sources;
e. a time-shared adaptive individualized noise cancellation module which reduces noise that may have overlapping frequency components with the target signals or is statistically correlated with the target signals;
f. a cyclic system reconfiguration module for signal separation which uses the rhythmic but non-synchronized nature of the target signals to identify the signal transmission channels iteratively and perform separation of signals in real-time;
whereby target signals from multiple sources that have similar stochastic and frequency features are physically separated both from each other and also from extraneous sources of noise that may be statistically correlated with or have overlapping frequency components with the target signals.

7. A method for continuously and automatically performing pattern recognition and diagnosis of target signals during monitoring comprising:
a. Providing a means for identifying and extracting multiple parameters to be used for characterize said target signals;
b. Providing a means for derivation of Individualized parameter distributions that are derived from data using stochastic analysis methods thereby defining individual baselines for diagnosis
c. Providing a means to dynamically capture the changes of the individualized key parameters dynamic sound pattern tracking by using stochastic processes for example a method of windowed averaging with gradual data discarding to track pattern changes;
d. Providing a means for optimally selecting diagnosis regions to maximize accuracy of diagnosis based for example on a stochastic optimization procedure that can use a multi-objective performance index to minimize combined errors thereby generating diagnosis regions accurately, individually, and objectively.
e. Providing a means for a recursive process which updates diagnosis regions when new data have been acquired thereby producing new parameters continuously;
f. Providing a means to provide electronic or visual display of the pattern recognition information and the motion path of extracted key parameters;
whereby affirmation for successful restriction of key parameters to a normal region, warning alarms for deviation of key parameters from their safe regions, and remedial recommendations based on the automated parameter trajectories can be provided to an observer of said display.

8. The method of claim 6 wherein said sensors may be acoustic or other types and the target signals may be body sounds or other types, such that rhythmic cycles of the signals are used wherein the method generates clarified signal attributes including among others the separation of heart from lung sounds.

9. The method of claim 6 wherein said time-shared and individualized noise cancellation noise and cyclic system reconfiguration signal separation methods are recursive and update noise channel models and achieve noise cancellation, cycle to cycle so that the processes are computationally very efficient since models are updated by using only new measurements and no past data needs to be stored and thereby dynamically adjusting to changes in the patient, environment, or both in real-time.

10. The method of claim 6 wherein said time-shared and individualized noise cancellation noise and cyclic system reconfiguration signal separation methods are preceded by traditional noise cancellation methods for example those that rely on frequency band separation, band-pass filters or stochastic separation such that the overall noise reduction is the most effective possible.

11. The method of claim 7 wherein said adaptive individualized pattern recognition and real-time individualized optimal diagnosis are recursive so that the processes can dynamically adjust to changes in the patient, environment, or both in real-time and with the minimum computational requirements.

12. The combined noise removal and signal separation method of claim 6 followed by the pattern recognition and diagnosis method of claim 7 wherein the combined overall method can automatically perform continuously noise reduction, target signal separation, pattern recognition, and diagnosis in real-time.

13. The combined noise removal and signal separation method of claim 8 followed by the pattern recognition and diagnosis method of claim 7 wherein the overall method can automatically perform continuous monitoring and diagnosis of body conditions and diseases in real-time.

14. The method of claim 7 wherein said target signals are body vital signs including for example of lung sounds and the characterizing variables may include, but are not limited to, inhale length and strength, exhale length and strength, breath cycle length, in the time domain; and center frequency, power, frequency bandwidth, for inhale and exhale individually, in the frequency domain.

15. The method of claim 12 wherein the diagnosis process identifies specific patterns for diseases, calculates individualized diagnosis algorithms, optimizes diagnosis regions, carries its tasks in real-time for specific disorders with quantitative confidence levels of diagnosis accuracy; and electronically displays the past medical conditions, current diagnosis, and near-future predictions of a patient's symptoms and disorders.
